(19) United States
(12) Patent Application Publication
(10) Pub. No.: US 2022/0346639 A1
(43) Pub. Date: Nov. 3, 2022

(54) SYSTEMS AND METHODS FOR THERMAL MANAGEMENT OF IMAGING DEVICES

(71) Applicant: Vicarious Surgical Inc., Waltham, MA (US)

(72) Inventors: Rolando Rodarte, Cambridge, MA (US); Justin Keenan, Lexington, MA (US); Marshall Wentworth, Waltham, MA (US)

(21) Appl. No.: 17/733,484

(22) Filed: Apr. 29, 2022

Related U.S. Application Data
(60) Provisional application No. 63/182,463, filed on Apr. 30, 2021.

(51) Int. Cl. A61B 1/12; C09D 5/24; C09D 127/18; C09D 7/61

(52) U.S. Cl. CPC A61B 1/128; C09D 5/24; C09D 127/18; C09D 7/61

(57) ABSTRACT

The present disclosure provides systems and methods for thermal management of an imaging device that is placed within a body of an individual during a medical procedure or a surgical procedure. In an aspect, the present disclosure provides an imaging device configured for use in a medical procedure or a surgical procedure while the imaging device is within a body of an individual undergoing the medical procedure or the surgical procedure. The imaging device may comprise a coating on at least a portion of an exterior of the imaging device, wherein the coating comprises a high thermal emissivity. The imaging device may comprise a set of thermal fins disposed on an exterior of the imaging device. The imaging device may comprise an endoscope.

### CROSS-REFERENCE

This application claims the benefit of U.S. Provisional Application No. 63/182,463, filed Apr. 30, 2021, which is entirely incorporated herein by reference.

### BACKGROUND

Numerous surgical procedures utilize endoscopes with cameras to visualize anatomical structures. Endoscopes may reach extremely high temperatures at the light-emitting part of the endoscope, due to light sources and camera drive electronics.

### SUMMARY

Described herein are systems and methods for thermal management of an endoscope that is placed within a body of an individual during a medical procedure or a surgical procedure.

Cameras and endoscopes used during medical procedure or surgical procedures may present numerous challenges in thermal management. Endoscopes may reach extremely high temperatures at the light-emitting part of the endoscope, due to light sources and camera drive electronics. Certain designs of flexible endoscopes, such as a chip-on-tip endoscope, may be more advantageous due to not needing flexible expensive optics.

Current endoscopes can reach extremely high temperatures at the light emitting part of the endoscope, with a risk of drapes catching on fire due to the emitting surface. Current endoscopes may not mitigate temperature risks, and may be operated with a common practice and understanding that long contact with the patient's anatomy is to be avoided during an endoscopy procedure. However, such practices involve risk of fire and risk of contact injury to the patient due to tissue burns. Further, reliance on the user to ensure safe operating margins carries risks due to user error or inattention. Further, in the case of surgical robotics or telemedicine, the surgeon is no longer located at the patient's bedside, which potentially makes the traditional use of endoscopes even more risky or disruptive to a medical procedure or a surgical procedure.

Some current endoscopes comprise low-power chips with CDVS to reduce thermal output in the device. However, such devices often have less than ideal performance and higher cost than their smartphone counterparts.

Recognizing the need for improved thermal management in imaging devices (e.g., endoscopes), the present disclosure provides systems and methods of thermal management in imaging devices (e.g., endoscopes), which may include the use of thermal coatings and/or thermal fins. The thermal coatings may comprise a high thermal emissivity. The coating may be a passive solution for reducing the surface temperature of the endoscope, which does not affect other parts of the design of the endoscope other than the color. The thermal fins may comprise mechanical fins disposed on a surface of the camera body.

Benefits of the systems and methods described herein generally include that such endoscopes with improved thermal management may be able to maintain a lower operating temperature (e.g., average or maximum operating temperature), and may be compatible with commercially available camera components for lower complexity and costs and increased performance.

In an aspect, the present disclosure provides an imaging device for use in a medical procedure or a surgical procedure within a body of an individual; wherein the imaging device comprises a coating on at least a portion of an exterior of the imaging device; and wherein the coating comprises a thermal emissivity of about 0.20 to 0.99. In some embodiments, the coating comprises a metal or metal alloy. In some embodiments, the coating comprises the metal. In some embodiments, the metal is selected from the group consisting of aluminum, magnesium, and titanium. In some embodiments, the imaging device comprises an endoscope. In some embodiments, the coating comprises polytetrafluoroethylene (PTFE). In some embodiments, the coating comprises more than one layer. In some embodiments, the more than one layer comprises an inner layer comprising a conductive material. In some embodiments, the conductive material comprises aluminum or copper or a combination thereof. In some embodiments, the more than one layer comprises an outer layer comprising a material with a higher thermal emissivity than the inner layer. In some embodiments, the material with a higher thermal emissivity comprises PEEK, glazed porcelain, or polypropylene or a combination thereof.

In another aspect, the present disclosure provides an imaging device for use in a medical procedure or a surgical procedure within a body of an individual; wherein the imaging device comprises a coating on at least a portion of an exterior of the imaging device; and wherein the imaging device is configured to dissipate heat at a rate of about 2 to 20 times as compared to a rate of heat dissipation of an imaging device not comprising the coating. In some embodiments, the coating comprises a metal or metal alloy. In some embodiments, the coating comprises the metal. In some embodiments, the metal is selected from the group consisting of aluminum, magnesium, and titanium. In some embodiments, the imaging device comprises an endoscope. In some embodiments, the coating comprises polytetrafluoroethylene (PTFE). In some embodiments, the coating comprises more than one layer. In some embodiments, the more than one layer comprises an inner layer comprising a conductive material. In some embodiments, the conductive material comprises aluminum or copper or a combination thereof. In some embodiments, the more than one layer comprises an outer layer comprising a material with a higher thermal emissivity than the inner layer. In some embodiments, the material with a higher thermal emissivity comprises PEEK, glazed porcelain, or polypropylene or a combination thereof

In another aspect, the present disclosure provides an imaging device for use in a medical procedure or a surgical procedure within a body of an individual; wherein the imaging device comprises a coating on at least a portion of an exterior of the imaging device; and wherein the imaging device operates at a maximum temperature of about 10° C. to 100° C. In some embodiments, the coating comprises a metal or metal alloy. In some embodiments, the coating comprises the metal. In some embodiments, the metal is selected from the group consisting of aluminum, magnesium, and titanium. In some embodiments, the imaging device comprises an endoscope. In some embodiments, the coating comprises polytetrafluoroethylene (PTFE). In some embodiments, the coating comprises more than one layer. In some embodiments, the more than one layer comprises an inner layer comprising a conductive material. In some embodiments, the conductive material comprises aluminum or copper or a combination thereof. In some embodiments, the more than one layer comprises an outer layer comprising a material with a higher thermal emissivity than the inner layer. In some embodiments, the material with a higher thermal emissivity comprises PEEK, glazed porcelain, or polypropylene or a combination thereof.

In another aspect, the present disclosure provides an imaging device for use in a medical procedure or a surgical procedure within a body of an individual; wherein the imaging device comprises a coating on at least a portion of an exterior of the imaging device; and wherein the imaging device is configured to allow a maximum contact time with the individual undergoing the medical procedure or the surgical procedure that is about 2 to 20 times as compared to a maximum contact time with the individual undergoing the medical procedure or the surgical procedure of an imaging device not comprising the coating. In some embodiments, the coating comprises a metal or metal alloy. In some embodiments, the coating comprises the metal. In some embodiments, the metal is selected from the group consisting of aluminum, magnesium, and titanium. In some embodiments, the imaging device comprises an endoscope. In some embodiments, the coating comprises polytetrafluoroethylene (PTFE). In some embodiments, the coating comprises more than one layer. In some embodiments, the more than one layer comprises an inner layer comprising a conductive material. In some embodiments, the conductive material comprises aluminum or copper or a combination thereof. In some embodiments, the more than one layer comprises an outer layer comprising a material with a higher thermal emissivity than the inner layer. In some embodiments, the material with a higher thermal emissivity comprises PEEK, glazed porcelain, or polypropylene or a combination thereof.

In another aspect, the present disclosure provides an imaging device for use in a medical procedure or a surgical procedure within a body of an individual; wherein the imaging device comprises a set of thermal fins disposed on an exterior of the imaging device; and wherein the set of thermal fins increases a surface area of the exterior of the imaging device by a factor of about 2 to 20 times as compared to an imaging device not comprising the set of thermal fins. In some embodiments, the imaging device comprises a coating comprising a metal or metal alloy. In some embodiments, the coating comprises the metal. In some embodiments, the metal is selected from the group consisting of aluminum, magnesium, and titanium. In some embodiments, the imaging device comprises an endoscope. In some embodiments, the imaging device comprises a polytetrafluoroethylene (PTFE) coating. In some embodiments, the imaging device comprises a coating comprising more than one layer. In some embodiments, the more than one layer comprises an inner layer comprising a conductive material. In some embodiments, the conductive material comprises aluminum or copper or a combination thereof. In some embodiments, the more than one layer comprises an outer layer comprising a material with a higher thermal emissivity than the inner layer. In some embodiments, the material with a higher thermal emissivity comprises PEEK, glazed porcelain, or polypropylene or a combination thereof. In some embodiments, the thermal fins comprise one or more of: vertical fins, radial fins, cylindrical fins, linear slots, or pin fins. In some embodiments, the pin fins are arranged in a grid pattern. In some embodiments, the thermal fins comprise at least 1, 2, 3, 4, 5, 6, 7, 8, 9, or 10 thermal fins.

In another aspect, the present disclosure provides an imaging device for use in a medical procedure or a surgical procedure within a body of an individual; wherein the imaging device comprises a set of thermal fins disposed on an exterior of the imaging device; and wherein the imaging device is configured to dissipate heat at a rate of about 2 to 20 times as compared to a rate of heat dissipation of an imaging device not comprising the set of thermal fins. In some embodiments, the imaging device comprises a coating comprising a metal or metal alloy. In some embodiments, the coating comprises the metal. In some embodiments, the metal is selected from the group consisting of aluminum, magnesium, and titanium. In some embodiments, the imaging device comprises an endoscope. In some embodiments, the imaging device comprises a polytetrafluoroethylene (PTFE) coating. In some embodiments, the imaging device comprises a coating comprising more than one layer. In some embodiments, the more than one layer comprises an inner layer comprising a conductive material. In some embodiments, the conductive material comprises aluminum or copper or a combination thereof. In some embodiments, the more than one layer comprises an outer layer comprising a material with a higher thermal emissivity than the inner layer. In some embodiments, the material with a higher thermal emissivity comprises PEEK, glazed porcelain, or polypropylene or a combination thereof. In some embodiments, the thermal fins comprise one or more of: vertical fins, radial fins, cylindrical fins, linear slots, or pin fins. In some embodiments, the pin fins are arranged in a grid pattern. In some embodiments, the thermal fins comprise at least 1, 2, 3, 4, 5, 6, 7, 8, 9, or 10 thermal fins.

In another aspect, the present disclosure provides an imaging device for use in a medical procedure or a surgical procedure within a body of an individual; wherein the imaging device comprises a set of thermal fins disposed on an exterior of the imaging device; and wherein the imaging device operates at a maximum temperature of about 10° C. to 100° C. In some embodiments, the coating comprises a metal or metal alloy. In some embodiments, the imaging device comprises a coating comprising the metal. In some embodiments, the metal is selected from the group consisting of aluminum, magnesium, and titanium. In some embodiments, the imaging device comprises an endoscope. In some embodiments, the imaging device comprises a polytetrafluoroethylene (PTFE) coating. In some embodiments, the imaging device comprises a coating comprising more than one layer. In some embodiments, the more than one layer comprises an inner layer comprising a conductive material. In some embodiments, the conductive material comprises aluminum or copper or a combination thereof. In some embodiments, the more than one layer comprises an outer layer comprising a material with a higher thermal emissivity than the inner layer. In some embodiments, the material with a higher thermal emissivity comprises PEEK, glazed porcelain, or polypropylene or a combination thereof. In some embodiments, the thermal fins comprise one or more of: vertical fins, radial fins, cylindrical fins, linear slots, or pin fins. In some embodiments, the pin fins are arranged in a grid pattern. In some embodiments, the thermal fins comprise at least 1, 2, 3, 4, 5, 6, 7, 8, 9, or 10 thermal fins.

In another aspect, the present disclosure provides an imaging device for use in a medical procedure or a surgical procedure within a body of an individual; wherein the imaging device comprises a set of thermal fins disposed on an exterior of the imaging device; and wherein the imaging device is configured to allow a maximum contact time with the individual undergoing the medical procedure or the surgical procedure that is about 2 to 20 times as compared to a maximum contact time with the individual undergoing the medical procedure or the surgical procedure of an imaging device not comprising the set of thermal fins. In some embodiments, the imaging device comprises a coating comprising metal or metal alloy. In some embodiments, the imaging devices comprises a coating comprising metal. In some embodiments, the metal is selected from the group consisting of aluminum, magnesium, and titanium. In some embodiments, the imaging device comprises an endoscope. In some embodiments, the coating comprises polytetrafluoroethylene (PTFE). In some embodiments, the imaging device comprises a coating comprising more than one layer. In some embodiments, the more than one layer comprises an inner layer comprising a conductive material. In some embodiments, the conductive material comprises aluminum or copper or a combination thereof. In some embodiments, the more than one layer comprises an outer layer comprising a material with a higher thermal emissivity than the inner layer. In some embodiments, the material with a higher thermal emissivity comprises PEEK, glazed porcelain, or polypropylene or a combination thereof. In some embodiments, the thermal fins comprise one or more of: vertical fins, radial fins, cylindrical fins, linear slots, or pin fins. In some embodiments, the pin fins are arranged in a grid pattern. In some embodiments, the thermal fins comprise at least 1, 2, 3, 4, 5, 6, 7, 8, 9, or 10 thermal fins.

In another aspect, the present disclosure provides a method for performing a medical procedure or a surgical procedure for an individual, comprising: (a) inserting at least a portion of an imaging device into a body of the individual; and (b) performing the medical procedure or the surgical procedure while the imaging device is within the body of the individual, wherein the imaging device comprises a coating on at least a portion of an exterior of the imaging device, and wherein the coating comprises a thermal emissivity of about 0.20 to 0.99. In some embodiments, the coating comprises a metal or metal alloy. In some embodiments, the coating comprises the metal. In some embodiments, the metal is selected from the group consisting of aluminum, magnesium, and titanium. In some embodiments, the imaging device comprises an endoscope. In some embodiments, the coating comprises polytetrafluoroethylene (PTFE). In some embodiments, the coating comprises more than one layer. In some embodiments, the more than one layer comprises an inner layer comprising a conductive material. In some embodiments, the conductive material comprises aluminum or copper or a combination thereof. In some embodiments, the more than one layer comprises an outer layer comprising a material with a higher thermal emissivity than the inner layer. In some embodiments, the material with a higher thermal emissivity comprises PEEK, glazed porcelain, or polypropylene or a combination thereof

In another aspect, the present disclosure provides a method for performing a medical procedure or a surgical procedure for an individual, comprising: (a) inserting at least a portion of an imaging device into a body of the individual; and (b) performing the medical procedure or the surgical procedure while the imaging device is within the body of the individual, wherein the imaging device comprises a coating on at least a portion of an exterior of the imaging device, and wherein the imaging device is configured to dissipate heat at a rate of about 2 to 20 times as compared to a rate of heat dissipation of an imaging device not comprising the coating. In some embodiments, the coating comprises a metal or metal alloy. In some embodiments, the coating comprises the metal. In some embodiments, the metal is selected from the group consisting of aluminum, magnesium, and titanium. In some embodiments, the imaging device comprises an endoscope. In some embodiments, the coating comprises polytetrafluoroethylene (PTFE). In some embodiments, the coating comprises more than one layer. In some embodiments, the more than one layer comprises an inner layer comprising a conductive material. In some embodiments, the conductive material comprises aluminum or copper or a combination thereof. In some embodiments, the more than one layer comprises an outer layer comprising a material with a higher thermal emissivity than the inner layer. In some embodiments, the material with a higher thermal emissivity comprises PEEK, glazed porcelain, or polypropylene or a combination thereof.

In another aspect, the present disclosure provides a method for performing a medical procedure or a surgical procedure for an individual, comprising: (a) inserting at least a portion of an imaging device into a body of the individual; and (b) performing the medical procedure or the surgical procedure while the imaging device is within the body of the individual, wherein the imaging device comprises a coating on at least a portion of an exterior of the imaging device, and wherein the imaging device operates at a maximum temperature of about 10° C. to 100° C. In some embodiments, the coating comprises a metal or metal alloy. In some embodiments, the coating comprises the metal. In some embodiments, the metal is selected from the group consisting of aluminum, magnesium, and titanium. In some embodiments, the imaging device comprises an endoscope. In some embodiments, the coating comprises polytetrafluoroethylene (PTFE). In some embodiments, the coating comprises more than one layer. In some embodiments, the more than one layer comprises an inner layer comprising a conductive material. In some embodiments, the conductive material comprises aluminum or copper or a combination thereof. In some embodiments, the more than one layer comprises an outer layer comprising a material with a higher thermal emissivity than the inner layer. In some embodiments, the material with a higher thermal emissivity comprises PEEK, glazed porcelain, or polypropylene or a combination thereof.

In another aspect, the present disclosure provides a method for performing a medical procedure or a surgical procedure for an individual, comprising: (a) inserting at least a portion of an imaging device into a body of the individual; and (b) performing the medical procedure or the surgical procedure while the imaging device is within the body of the individual, wherein the imaging device comprises a coating on at least a portion of an exterior of the imaging device, and wherein the imaging device is configured to allow a maximum contact time with the individual undergoing the medical procedure or the surgical procedure that is about 2 to 20 times as compared to a maximum contact time with the individual undergoing the medical procedure or the surgical procedure of an imaging device not comprising the coating. In some embodiments, the coating comprises a metal or metal alloy. In some embodiments, the coating comprises the metal. In some embodiments, the metal is selected from the group consisting of aluminum, magnesium, and titanium. In some embodiments, the imaging device comprises an endoscope. In some embodiments, the coating comprises polytetrafluoroethylene (PTFE). In some embodiments, the coating comprises more than one layer. In some embodiments, the more than one layer comprises an inner layer comprising a conductive material. In some embodiments, the conductive material comprises aluminum or copper or a combination thereof. In some embodiments, the more than one layer comprises an outer layer comprising a material with a higher thermal emissivity than the inner layer. In some embodiments, the material with a higher thermal emissivity comprises PEEK, glazed porcelain, or polypropylene or a combination thereof.

In another aspect, the present disclosure provides a method for performing a medical procedure or a surgical procedure for an individual, comprising: (a) inserting at least a portion of an imaging device into a body of the individual; and (b) performing the medical procedure or the surgical procedure while the imaging device is within the body of the individual, wherein the imaging device comprises a set of thermal fins disposed on an exterior of the imaging device, and wherein the set of thermal fins increases a surface area of the exterior of the imaging device by a factor of about 2 to 20 times as compared to an imaging device not comprising the set of thermal fins. In some embodiments, the imaging device comprises a metal or metal alloy. In some embodiments, the imaging device comprises the metal. In some embodiments, the metal is selected from the group consisting of aluminum, magnesium, and titanium. In some embodiments, the imaging device comprises an endoscope. In some embodiments, the imaging device comprises a coating comprising polytetrafluoroethylene (PTFE). In some embodiments, the imaging device comprises a coating comprising more than one layer. In some embodiments, the more than one layer comprises an inner layer comprising a conductive material. In some embodiments, the conductive material comprises aluminum or copper or a combination thereof. In some embodiments, the more than one layer comprises an outer layer comprising a material with a higher thermal emissivity than the inner layer. In some embodiments, the material with a higher thermal emissivity comprises PEEK, glazed porcelain, or polypropylene or a combination thereof. In some embodiments, the thermal fins comprise one or more of: vertical fins, radial fins, cylindrical fins, linear slots, or pin fins. In some embodiments, the pin fins are arranged in a grid pattern. In some embodiments, the thermal fins comprise at least 1, 2, 3, 4, 5, 6, 7, 8, 9, or 10 thermal fins.

In another aspect, the present disclosure provides a method for performing a medical procedure or a surgical procedure for an individual, comprising: (a) inserting at least a portion of an imaging device into a body of the individual; and (b) performing the medical procedure or the surgical procedure while the imaging device is within the body of the individual, wherein the imaging device comprises a set of thermal fins disposed on an exterior of the imaging device, and wherein the imaging device is configured to dissipate heat at a rate of about 2 to 20 times as compared to a rate of heat dissipation of an imaging device not comprising the set of thermal fins. In some embodiments, the imaging device comprises a metal or metal alloy. In some embodiments, the imaging device comprises the metal. In some embodiments, the metal is selected from the group consisting of aluminum, magnesium, and titanium. In some embodiments, the imaging device comprises an endoscope. In some embodiments, the imaging device comprises a coating comprising polytetrafluoroethylene (PTFE). In some embodiments, the imaging device comprises a coating comprising more than one layer. In some embodiments, the more than one layer comprises an inner layer comprising a conductive material. In some embodiments, the conductive material comprises aluminum or copper or a combination thereof. In some embodiments, the more than one layer comprises an outer layer comprising a material with a higher thermal emissivity than the inner layer. In some embodiments, the material with a higher thermal emissivity comprises PEEK, glazed porcelain, or polypropylene or a combination thereof. In some embodiments, the thermal fins comprise one or more of: vertical fins, radial fins, cylindrical fins, linear slots, or pin fins. In some embodiments, the pin fins are arranged in a grid pattern. In some embodiments, the thermal fins comprise at least 1, 2, 3, 4, 5, 6, 7, 8, 9, or 10 thermal fins.

In another aspect, the present disclosure provides a method for performing a medical procedure or a surgical procedure for an individual, comprising: (a) inserting at least a portion of an imaging device into a body of the individual; and (b) performing the medical procedure or the surgical procedure while the imaging device is within the body of the individual, wherein the imaging device comprises a set of thermal fins disposed on an exterior of the imaging device, and wherein the imaging device operates at a maximum temperature of about 10° C. to 100° C. In some embodiments, the imaging device comprises a metal or metal alloy. In some embodiments, the imaging device comprises the metal. In some embodiments, the metal is selected from the group consisting of aluminum, magnesium, and titanium. In some embodiments, the imaging device comprises an endoscope. In some embodiments, the imaging device comprises a coating comprising polytetrafluoroethylene (PTFE). In some embodiments, the imaging device comprises a coating comprising more than one layer. In some embodiments, the more than one layer comprises an inner layer comprising a conductive material. In some embodiments, the conductive material comprises aluminum or copper or a combination thereof. In some embodiments, the more than one layer comprises an outer layer comprising a material with a high thermal emissivity. In some embodiments, the material with a high thermal emissivity comprises PEEK, glazed porcelain, or polypropylene or a combination thereof. In some embodiments, the thermal fins comprise one or more of: vertical fins, radial fins, cylindrical fins, linear slots, or pin fins. In some embodiments, the pin fins are arranged in a grid pattern. In some embodiments, the thermal fins comprise at least 1, 2, 3, 4, 5, 6, 7, 8, 9, or 10 thermal fins.

In another aspect, the present disclosure provides a method for performing a medical procedure or a surgical procedure for an individual, comprising: (a) inserting at least a portion of an imaging device into a body of the individual; and (b) performing the medical procedure or the surgical procedure while the imaging device is within the body of the individual, wherein the imaging device comprises a set of thermal fins disposed on an exterior of the imaging device, and wherein the imaging device is configured to allow a maximum contact time with the individual undergoing the medical procedure or the surgical procedure that is about 2 to 20 times as compared to a maximum contact time with the individual undergoing the medical procedure or the surgical procedure of an imaging device not comprising the set of thermal fins. In some embodiments, the imaging device comprises a metal or metal alloy. In some embodiments, the imaging device comprises the metal. In some embodiments, the metal is selected from the group consisting of aluminum, magnesium, and titanium. In some embodiments, the imaging device comprises an endoscope. In some embodiments, the imaging device comprises a coating comprising polytetrafluoroethylene (PTFE). In some embodiments, the imaging device comprises a coating comprising more than one layer. In some embodiments, the more than one layer comprises an inner layer comprising a conductive material. In some embodiments, the conductive material comprises aluminum or copper or a combination thereof. In some embodiments, the more than one layer comprises an outer layer comprising a material with a higher thermal emissivity than the inner layer. In some embodiments, the material with a higher thermal emissivity comprises PEEK, glazed porcelain, or polypropylene or a combination thereof. In some embodiments, the thermal fins comprise one or more of: vertical fins, radial fins, cylindrical fins, linear slots, or pin fins. In some embodiments, the pin fins are arranged in a grid pattern. In some embodiments, the thermal fins comprise at least 1, 2, 3, 4, 5, 6, 7, 8, 9, or 10 thermal fins.

In another aspect, the present disclosure provides a method of increasing thermal performance of an imaging device configured for use in a medical procedure or a surgical procedure while the imaging device is within a body of an individual undergoing the medical procedure or the surgical procedure, wherein the method comprises disposing a coating onto at least a portion of an exterior of the imaging device, wherein the coating comprises a thermal emissivity of about 0.20 to 0.99. In some embodiments, the coating comprises a metal or metal alloy. In some embodiments, the coating comprises the metal. In some embodiments, the metal is selected from the group consisting of aluminum, magnesium, and titanium. In some embodiments, the imaging device comprises an endoscope. In some embodiments, the coating comprises polytetrafluoroethylene (PTFE). In some embodiments, the coating comprises more than one layer. In some embodiments, the more than one layer comprises an inner layer comprising a conductive material. In some embodiments, the conductive material comprises aluminum or copper or a combination thereof. In some embodiments, the more than one layer comprises an outer layer comprising a material with a higher thermal emissivity than the inner layer. In some embodiments, the material with a higher thermal emissivity comprises PEEK, glazed porcelain, or polypropylene or a combination thereof.

In another aspect, the present disclosure provides a method of increasing thermal performance of an imaging device configured for use in a medical procedure or a surgical procedure while the imaging device is within a body of an individual undergoing the medical procedure or the surgical procedure, wherein the method comprises disposing a coating onto at least a portion of an exterior of the imaging device, thereby increasing a rate of heat dissipation of the imaging device by a factor of about 2 to 20 times. In some embodiments, the coating comprises a metal or metal alloy. In some embodiments, the coating comprises the metal. In some embodiments, the anodized metal is selected from the group consisting of aluminum, magnesium, and titanium. In some embodiments, the imaging device comprises an endoscope. In some embodiments, the coating comprises polytetrafluoroethylene (PTFE). In some embodiments, the coating comprises more than one layer. In some embodiments, the more than one layer comprises an inner layer comprising a conductive material. In some embodiments, the conductive material comprises aluminum or copper or a combination thereof. In some embodiments, the more than one layer comprises an outer layer comprising a material with a high thermal emissivity. In some embodiments, the material with a high thermal emissivity comprises PEEK, glazed porcelain, or polypropylene or a combination thereof.

In another aspect, the present disclosure provides a method of increasing thermal performance of an imaging device configured for use in a medical procedure or a surgical procedure while the imaging device is within a body of an individual undergoing the medical procedure or the surgical procedure, wherein the method comprises disposing a coating onto at least a portion of an exterior of the imaging device, thereby increasing a rate of heat dissipation of the imaging device sufficient for the imaging device to operate at a maximum temperature of about 10° C. to 100° C. In some embodiments, the coating comprises a metal or metal alloy. In some embodiments, the coating comprises the metal. In some embodiments, the metal is selected from the group consisting of aluminum, magnesium, and titanium. In some embodiments, the imaging device comprises an endoscope. In some embodiments, the coating comprises polytetrafluoroethylene (PTFE). In some embodiments, the coating comprises more than one layer. In some embodiments, the more than one layer comprises an inner layer comprising a conductive material. In some embodiments, the conductive material comprises aluminum or copper or a combination thereof. In some embodiments, the more than one layer comprises an outer layer comprising a material with a high thermal emissivity. In some embodiments, the material with a high thermal emissivity comprises PEEK, glazed porcelain, or polypropylene or a combination thereof.

In another aspect, the present disclosure provides a method of increasing thermal performance of an imaging device configured for use in a medical procedure or a surgical procedure while the imaging device is within a body of an individual undergoing the medical procedure or the surgical procedure, wherein the method comprises disposing a coating onto at least a portion of an exterior of the imaging device, thereby increasing a rate of heat dissipation of the imaging device sufficient for the imaging device to allow a maximum contact time with the individual undergoing the medical procedure or the surgical procedure that is increased by a factor of about 2 to 20 times. In some embodiments, the coating comprises a metal or metal alloy. In some embodiments, the coating comprises the metal. In some embodiments, the anodized metal is selected from the group consisting of aluminum, magnesium, and titanium. In some embodiments, the imaging device comprises an endoscope. In some embodiments, the coating comprises polytetrafluoroethylene (PTFE). In some embodiments, the coating comprises more than one layer. In some embodiments, the more than one layer comprises an inner layer comprising a conductive material. In some embodiments, the conductive material comprises aluminum or copper or a combination thereof. In some embodiments, the more than one layer comprises an outer layer comprising a material with a higher thermal emissivity than the inner layer. In some embodiments, the material with a higher thermal emissivity comprises PEEK, glazed porcelain, or polypropylene or a combination thereof.

In another aspect, the present disclosure provides a method of increasing thermal performance of an imaging device configured for use in a medical procedure or a surgical procedure while the imaging device is within a body of an individual undergoing the medical procedure or the surgical procedure, wherein the method comprises disposing a set of thermal fins on an exterior of the imaging device, thereby increasing a surface area of the exterior of the imaging device by a factor of at least about 2 times, at least about 3 times, at least about 4 times, at least about 5 times, at least about 6 times, at least about 7 times, at least about 8 times, at least about 9 times, at least about 10 times, at least about 11 times, at least about 12 times, at least about 13 times, at least about 14 times, at least about 15 times, at least about 16 times, at least about 17 times, at least about 18 times, at least about 19 times, or at least about 20 times. In some embodiments, the imaging device comprises a metal or metal alloy. In some embodiments, the imaging device comprises the metal. In some embodiments, the metal is selected from the group consisting of aluminum, magnesium, and titanium. In some embodiments, the imaging device comprises an endoscope. In some embodiments, the imaging device comprises a coating comprising polytetrafluoroethylene (PTFE). In some embodiments, the imaging device comprises a coating comprising more than one layer. In some embodiments, the more than one layer comprises an inner layer comprising a conductive material. In some embodiments, the conductive material comprises aluminum or copper or a combination thereof. In some embodiments, the more than one layer comprises an outer layer comprising a material with a higher thermal emissivity than the inner layer. In some embodiments, the material with a higher thermal emissivity comprises PEEK, glazed porcelain, or polypropylene or a combination thereof. In some embodiments, the thermal fins comprise one or more of: vertical fins, radial fins, cylindrical fins, linear slots, or pin fins. In some embodiments, the pin fins are arranged in a grid pattern. In some embodiments, the thermal fins comprise at least 1, 2, 3, 4, 5, 6, 7, 8, 9, or 10 thermal fins.

In another aspect, the present disclosure provides a method of increasing thermal performance of an imaging device configured for use in a medical procedure or a surgical procedure while the imaging device is within a body of an individual undergoing the medical procedure or the surgical procedure, wherein the method comprises disposing a set of thermal fins on an exterior of the imaging device, thereby increasing a rate of heat dissipation of the imaging device by a factor of about 2 to 20 times. In some embodiments, the imaging device comprises a metal or metal alloy. In some embodiments, the imaging device comprises the metal. In some embodiments, the metal is selected from the group consisting of aluminum, magnesium, and titanium. In some embodiments, the imaging device comprises an endoscope. In some embodiments, the imaging device comprises a coating comprising polytetrafluoroethylene (PTFE). In some embodiments, the imaging device comprises a coating comprising more than one layer. In some embodiments, the more than one layer comprises an inner layer comprising a conductive material. In some embodiments, the conductive material comprises aluminum or copper or a combination thereof. In some embodiments, the more than one layer comprises an outer layer comprising a material with a high thermal emissivity. In some embodiments, the material with a high thermal emissivity comprises PEEK, glazed porcelain, or polypropylene or a combination thereof. In some embodiments, the thermal fins comprise one or more of: vertical fins, radial fins, cylindrical fins, linear slots, or pin fins. In some embodiments, the pin fins are arranged in a grid pattern. In some embodiments, the thermal fins comprise at least 1, 2, 3, 4, 5, 6, 7, 8, 9, or 10 thermal fins.

In another aspect, the present disclosure provides a method of increasing thermal performance of an imaging device configured for use in a medical procedure or a surgical procedure while the imaging device is within a body of an individual undergoing the medical procedure or the surgical procedure, wherein the method comprises disposing a set of thermal fins on an exterior of the imaging device, thereby increasing a rate of heat dissipation of the imaging device sufficient for the imaging device to operate at a maximum temperature of about 10° C. to 100° C. In some embodiments, the imaging device comprises a metal or metal alloy. In some embodiments, the imaging device comprises the metal. In some embodiments, the metal is selected from the group consisting of aluminum, magnesium, and titanium. In some embodiments, the imaging device comprises an endoscope. In some embodiments, the imaging device comprises a coating comprising polytetrafluoroethylene (PTFE). In some embodiments, the imaging device comprises a coating comprising more than one layer. In some embodiments, the more than one layer comprises an inner layer comprising a conductive material. In some embodiments, the conductive material comprises aluminum or copper or a combination thereof. In some embodiments, the more than one layer comprises an outer layer comprising a material with a high thermal emissivity. In some embodiments, the material with a high thermal emissivity comprises PEEK, glazed porcelain, or polypropylene or a combination thereof. In some embodiments, the thermal fins comprise one or more of: vertical fins, radial fins, cylindrical fins, linear slots, or pin fins. In some embodiments, the pin fins are arranged in a grid pattern. In some embodiments, the thermal fins comprise at least 1, 2, 3, 4, 5, 6, 7, 8, 9, or 10 thermal fins.

In another aspect, the present disclosure provides a method of increasing thermal performance of an imaging device configured for use in a medical procedure or a surgical procedure while the imaging device is within a body of an individual undergoing the medical procedure or the surgical procedure, wherein the method comprises disposing a set of thermal fins on an exterior of the imaging device, thereby increasing a rate of heat dissipation of the imaging device sufficient for the imaging device to allow a maximum contact time with the individual undergoing the medical procedure or the surgical procedure that is increased by a factor of about 2 to 20 times. In some embodiments, the imaging device comprises a metal or metal alloy. In some embodiments, the imaging device comprises the metal. In some embodiments, the metal is selected from the group consisting of aluminum, magnesium, and titanium. In some embodiments, the imaging device comprises an endoscope. In some embodiments, the imaging device comprises a coating comprising polytetrafluoroethylene (PTFE). In some embodiments, the imaging device comprises a coating comprising more than one layer. In some embodiments, the more than one layer comprises an inner layer comprising a conductive material. In some embodiments, the conductive material comprises aluminum or copper or a combination thereof. In some embodiments, the more than one layer comprises an outer layer comprising a material with a high thermal emissivity. In some embodiments, the material with a high thermal emissivity comprises PEEK, glazed porcelain, or polypropylene or a combination thereof. In some embodiments, the thermal fins comprise one or more of: vertical fins, radial fins, cylindrical fins, linear slots, or pin fins. In some embodiments, the pin fins are arranged in a grid pattern. In some embodiments, the thermal fins comprise at least 1, 2, 3, 4, 5, 6, 7, 8, 9, or 10 thermal fins.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an example of two endoscopes, including a top endoscope having a thermal coating and a bottom endoscope not having a thermal coating.

FIG. 2 shows an example of a camera with thermal fins visible on the extremes of the camera.

FIG. 3 shows an example of thermal fins of an endoscope. Two sections of vertical fins and radial fins are used to maximize surface area of the module.

FIG. 4 shows an example of linear slots that allow reduced temperature at the contact surfaces but not substantially increased overall temperature.

FIG. 5 shows a front view of an example of a camera having thermal fins visible on the extremes of the camera.

FIG. 6 shows a perspective view of exemplary embodiments of a plurality of cylindrical thermal fins.

FIG. 7 shows a side view of exemplary embodiments of a plurality of cylindrical thermal fins.

FIG. 8 shows an exemplary embodiment of an isometric view of the imaging device comprising a plurality of thermal fins.

FIG. 9 shows a perspective view of exemplary embodiments of a plurality of vertical thermal fins separated by a plurality of linear slots.

FIG. 10 shows a front view of exemplary embodiments of a plurality of vertical thermal fins separated by a plurality of linear slots.

FIG. 11 shows a perspective view of exemplary embodiments of a plurality of thermal fins in a grid pattern.

FIG. 12 shows a perspective view of exemplary embodiments of a plurality of thermal fins separated by a plurality of linear slots.

FIG. 13 shows a right perspective view of the plurality of thermal fins separated by the plurality of linear slots.

FIG. 14 shows a left perspective view of the plurality of thermal fins separated by the plurality of linear slots.

FIG. 15 shows a front view of the plurality of thermal fins separated by the plurality of linear slots.

FIG. 16 shows a side view of the plurality of thermal fins separated by the plurality of linear slots.

FIG. 17 shows a side view of plurality of thermal fins, including a plurality of cylindrical thermal fins and a plurality of thermal fins separated by the plurality of linear slots.

### DETAILED DESCRIPTION OF THE INVENTION

Numerous surgical procedures utilize endoscopes with cameras to visualize anatomical structures. Endoscopes may reach extremely high temperatures at the light-emitting part of the endoscope, due to light sources and camera drive electronics. Cameras and endoscopes used during medical procedure or surgical procedures may present numerous challenges in thermal management. Endoscopes may reach extremely high temperatures at the light-emitting part of the endoscope, due to light sources and camera drive electronics. Certain designs of flexible endoscopes, such as a chip-on-tip endoscope, may be more advantageous due to not needing flexible expensive optics.

Current endoscopes can reach extremely high temperatures at the light emitting part of the endoscope, with a risk of drapes catching on fire due to the emitting surface. Current endoscopes may not mitigate temperature risks, and may be operated with a common practice and understanding that long contact with the patient's anatomy is to be avoided during an endoscopy procedure. However, such practices involve risk of fire and risk of contact injury to the patient due to tissue burns. Further, reliance on the user to ensure safe operating margins carries risks due to user error or inattention. Further, in the case of surgical robotics or telemedicine, the surgeon is no longer located at the patient's bedside, which potentially makes the traditional use of endoscopes even more risky or disruptive to a medical procedure or a surgical procedure.

Some current endoscopes comprise low-power chips with CDVS to reduce thermal output in the device. However, such devices often have less than ideal imaging performance and higher cost than their smartphone counterparts.

Described herein are systems and methods for thermal management of an endoscope that is placed within a body of an individual during a medical procedure or a surgical procedure. Recognizing the need for improved thermal management in imaging devices (e.g., endoscopes), the present disclosure provides systems and methods of thermal management in imaging devices (e.g., endoscopes), which may include the use of thermal coatings and/or thermal fins. The thermal coatings may comprise a high thermal emissivity. The coating may be a passive solution for reducing the surface temperature of the endoscope, which does not affect other parts of the design of the endoscope other than the color. The thermal fins may comprise mechanical fins disposed on a surface of the camera body.

Benefits of the systems and methods described herein generally include that such endoscopes with improved thermal management may be able to maintain a lower operating temperature (e.g., average or maximum operating temperature), and may be compatible with commercially available camera components for lower complexity and costs and increased performance.

In an aspect, the present disclosure provides an imaging device configured for use in a medical procedure or a surgical procedure while the imaging device is within a body of an individual undergoing the medical procedure or the surgical procedure; wherein the imaging device comprises a coating on at least a portion of an exterior of the imaging device; and wherein the coating comprises a thermal emissivity of at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.97, at least about 0.98, or at least about 0.99.

In some embodiments, at least a portion of the exterior surface of the imaging device comprises a coating. In some embodiments, the coating is on the exterior surface of the imaging device. In some embodiments, the lens of the imaging device does not have a coating in order to allow for clear imaging. In some embodiments, the coating comprises a metal or metal alloy. In some embodiments, the coating comprises the metal. In some embodiments, the metal is an anodized metal. In some embodiments, the anodization is type III anodization that can be applied to aluminum, steel and titanium, and polytetrafluoroethylene (PTFE). In some embodiments, the anodized metal is selected from the group consisting of aluminum, magnesium, and titanium. In some embodiments, the anodized metal is the aluminum. In some embodiments, the anodized metal is the magnesium. In some embodiments, the anodized metal is the titanium. In some embodiments, the coating comprises the metal alloy. In some embodiments, the metal alloy comprises a metal selected from the group consisting of aluminum, magnesium, and titanium. In some embodiments, the metal is the aluminum. In some embodiments, the metal is the magnesium. In some embodiments, the metal is the titanium. In some embodiments, the coating comprises a polymer. In some embodiments the polymeric coating comprises polytetrafluoroethylene (PTFE). In some embodiments, the PTFE comprises Magic Black, Vantablack, or other engineered coatings. In some embodiments, the PTFE coatings have thermal emissivities ranging from 0.5 to 1, 0.7 to 1, 0.8 to 1, or 0.85 to 1. In some embodiments, the imaging device comprises an endoscope.

In some embodiments, the coating comprises more than one layer. In some embodiments, the coating comprises a double layer. In some embodiments, the more than one layer of coating comprises more than one material. In some embodiments, the coating comprises a composite material. In some embodiments, a coating comprising a composite material may be preferred in a bigger application. In some embodiments, the coating comprises an inner layer and an outer layer. In some embodiments, the coating comprises a conductive material as an inner layer and a thin outer material. In some embodiments, the conductive material comprises aluminum or copper. In some embodiments, the outer layer comprises a material with a high emissivity, including but not limited to PEEK. In some embodiments, the material with a high emissivity increases the thermal load that the imaging device can dissipate to the environment.

Provided herein are various approaches to increase the thermal emissivity to enhance thermal performance of imaging devices. In some embodiments, the approaches to increase the thermal emissivity include but are not limited to using different materials, composite materials, and/or different coatings. In some embodiments, the imaging device comprises a material with a high thermal emissivity. In some embodiments, the material with a high thermal emissivity does not need coatings for thermal management. In some embodiments, the material with a high thermal emissivity comprises plastics. In some embodiments, the material with a high thermal emissivity comprises PEEK, glazed porcelain, and polypropylene. In some embodiments, the material with a high thermal emissivity has a thermal emissivity ranging from 0.5 to 1, 0.6 to 1, 0.7 to 1, 0.8 to 1, 0.85 to 1, 0.9 to 1. In some embodiments, PEEK has a thermal emissivity of about 0.95. In some embodiments, glazed porcelain has a thermal emissivity of about 0.92. In some embodiments, polypropylene has a thermal emissivity of about 0.97.

In another aspect, the present disclosure provides an imaging device configured for use in a medical procedure or a surgical procedure while the imaging device is within a body of an individual undergoing the medical procedure or the surgical procedure; wherein the imaging device comprises a coating on at least a portion of an exterior of the imaging device; and wherein the imaging device is configured to dissipate heat at a rate of at least about 2 times, at least about 3 times, at least about 4 times, at least about 5 times, at least about 6 times, at least about 7 times, at least about 8 times, at least about 9 times, at least about 10 times, at least about 11 times, at least about 12 times, at least about 13 times, at least about 14 times, at least about 15 times, at least about 16 times, at least about 17 times, at least about 18 times, at least about 19 times, or at least about 20 times, as compared to a rate of heat dissipation of an imaging device not comprising the coating. In some embodiments, the coating allows for a faster dissipation of thermal energy from the imaging device than without the coating.

In another aspect, the present disclosure provides an imaging device configured for use in a medical procedure or a surgical procedure while the imaging device is within a body of an individual undergoing the medical procedure or the surgical procedure; wherein the imaging device comprises a coating on at least a portion of an exterior of the imaging device; and wherein the imaging device operates at a maximum temperature of no more than about 100° C., no more than about 95° C., no more than about 90° C., no more than about 85° C., no more than about 80° C., no more than about 75° C., no more than about 70° C., no more than about 65° C., no more than about 60° C., no more than about 55° C., no more than about 50° C., no more than about 45° C., no more than about 40° C., no more than about 35° C., no more than about 30° C., no more than about 25° C., no more than about 20° C., no more than about 15° C., or no more than about 10° C.

In another aspect, the present disclosure provides an imaging device configured for use in a medical procedure or a surgical procedure while the imaging device is within a body of an individual undergoing the medical procedure or the surgical procedure; wherein the imaging device comprises a coating on at least a portion of an exterior of the imaging device; and wherein the imaging device is configured to allow a maximum contact time with the individual undergoing the medical procedure or the surgical procedure that is at least about 2 times, at least about 3 times, at least about 4 times, at least about 5 times, at least about 6 times, at least about 7 times, at least about 8 times, at least about 9 times, at least about 10 times, at least about 11 times, at least about 12 times, at least about 13 times, at least about 14 times, at least about 15 times, at least about 16 times, at least about 17 times, at least about 18 times, at least about 19 times, or at least about 20 times, as compared to a maximum contact time with the individual undergoing the medical procedure or the surgical procedure of an imaging device not comprising the coating.

In another aspect, the present disclosure provides an imaging device configured for use in a medical procedure or a surgical procedure while the imaging device is within a body of an individual undergoing the medical procedure or the surgical procedure; wherein the imaging device comprises a set of thermal fins disposed on an exterior of the imaging device; and wherein the set of thermal fins increases a surface area of the exterior of the imaging device by a factor of at least about 2 times, at least about 3 times, at least about 4 times, at least about 5 times, at least about 6 times, at least about 7 times, at least about 8 times, at least about 9 times, at least about 10 times, at least about 11 times, at least about 12 times, at least about 13 times, at least about 14 times, at least about 15 times, at least about 16 times, at least about 17 times, at least about 18 times, at least about 19 times, or at least about 20 times, as compared to an imaging device not comprising the set of thermal fins.

In some embodiments, the thermal fins increase the surface areas of the device in contact with the environment. In some embodiments, the increase in surface area may result in an increased heat transfer from the imaging device to the environment. In some embodiments, the thermal fins comprise one or more protrusions from the exterior surface of the imaging device. In some embodiments, the protrusion extends from a housing of the imaging device. In some embodiments, the thermal fins comprise one or more elongated protrusions from the exterior surface of the imaging device. In some embodiments, the thermal fins comprise a plurality of protrusions in a grid pattern.

In some embodiments, the thermal fins allow for a robust heat transfer even when the imaging device generates heat.

In some embodiments, the thermal fins comprise one or more elongated protrusions from the exterior surface of the imaging device. In some embodiments, the elongated protrusion is parallel to a long axis of the imaging device. In some embodiments, the one or more elongated protrusions are parallel to the long axis of a cover of the imaging device. In some embodiments, the one or more elongated protrusions are perpendicular to the long axis of the cover of the imaging device. Usually, the elongated protrusions provide various advantages, including but not limited to that the elongated protrusions are cooler than the rest of the imaging device. In some embodiments, the lower temperature of the thermal fins allows for longer duration of contact with tissue without causing damage. In some embodiments, the lower temperature of the thermal fins is due to the length of the fins. In some embodiments, the thermal efficiency of the thermal fin may be lower due to the lower temperature at which the thermal fins operate. In some embodiments, the lower thermal efficiency of the thermal fin results in less thermal transfer between the imaging device and the environment.

In some embodiments, the thermal fins comprise a plurality of protrusions in a grid pattern. In some embodiments, a protrusion in the plurality of protrusions is evenly spaced apart from a second protrusion. In some embodiments, the thermal fins allow for a more robust heat transfer even when the imaging device is not positioned at an optimal orientation with gravity compared to the vertical fin design due to the grid pattern. In some embodiments, when the imaging device is positioned in an optimal orientation for imaging, the heat transfer from the imaging device to the environment may be less efficient as the thermal fins may be stacked on top of each other.

In some embodiments, the thermal fins are sized to have a high surface area to volume ratio. In some embodiments, the ratio of surface area to volume is at least 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, or 500. In some embodiments, the ratio of surface area to volume ranges from 1 to 500, 1 to 400, 1 to 300, 1 to 200, 1 to 100, or 5 to 50.

In some embodiments, the thermal fins comprise a coating to facilitate thermal management. In some embodiments, the coating comprises a metal or metal alloy. In some embodiments, the coating comprises the metal. In some embodiments, the metal is an anodized metal. In some embodiments, the anodized metal is selected from the group consisting of aluminum, magnesium, and titanium. In some embodiments, the anodized metal is the aluminum. In some embodiments, the anodized metal is the magnesium. In some embodiments, the anodized metal is the titanium. In some embodiments, the coating comprises the metal alloy. In some embodiments, the metal alloy comprises a metal selected from the group consisting of aluminum, magnesium, and titanium. In some embodiments, the metal is the aluminum. In some embodiments, the metal is the magnesium. In some embodiments, the metal is the titanium. In some embodiments, the imaging device comprises an endoscope.

In some embodiments, the coating of the thermal fins comprises more than one layer. In some embodiments, the coating comprises a double layer. In some embodiments, the more than one layer of coating comprises more than one material. In some embodiments, the coating comprises a composite material. In some embodiments, a coating comprising a composite material may be preferred in a bigger application. In some embodiments, the coating comprises an inner layer and an outer layer. In some embodiments, the coating comprises a conductive material as an inner layer and a thin outer material. In some embodiments, the conductive material comprises aluminum or copper. In some embodiments, the outer layer comprises a material with a high emissivity, including but not limited to PEEK. In some embodiments, the material with a high emissivity increases the thermal load that the imaging device can dissipate to the environment.

Provided herein are various approaches to increase the thermal emissivity to enhance thermal performance of imaging devices. In some embodiments, the approaches to increase the thermal emissivity include but are not limited to using different materials, composite materials, and/or different coatings for the thermal fins. In some embodiments, the thermal fins comprise a material with a high thermal emissivity. In some embodiments, the material with a high thermal emissivity does not need coatings for thermal management. In some embodiments, the material with a high thermal emissivity comprises plastics. In some embodiments, the material with a high thermal emissivity comprises PEEK, glazed porcelain, and polypropylene. In some embodiments, the material with a high thermal emissivity has a thermal emissivity ranging from 0.5 to 1, 0.6 to 1, 0.7 to 1, 0.8 to 1, 0.85 to 1, 0.9 to 1. In some embodiments, PEEK has a thermal emissivity of about 0.95. In some embodiments, glazed porcelain has a thermal emissivity of about 0.92. In some embodiments, polypropylene has a thermal emissivity of about 0.97.

In another aspect, the present disclosure provides an imaging device configured for use in a medical procedure or a surgical procedure while the imaging device is within a body of an individual undergoing the medical procedure or the surgical procedure; wherein the imaging device comprises a set of thermal fins disposed on an exterior of the imaging device; and wherein the imaging device is configured to dissipate heat at a rate of at least about 2 times, at least about 3 times, at least about 4 times, at least about 5 times, at least about 6 times, at least about 7 times, at least about 8 times, at least about 9 times, at least about 10 times, at least about 11 times, at least about 12 times, at least about 13 times, at least about 14 times, at least about 15 times, at least about 16 times, at least about 17 times, at least about 18 times, at least about 19 times, or at least about 20 times, as compared to a rate of heat dissipation of an imaging device not comprising the set of thermal fins.

In another aspect, the present disclosure provides an imaging device configured for use in a medical procedure or a surgical procedure while the imaging device is within a body of an individual undergoing the medical procedure or the surgical procedure; wherein the imaging device comprises a set of thermal fins disposed on an exterior of the imaging device; and wherein the imaging device operates at a maximum temperature of no more than about 100° C., no more than about 95° C., no more than about 90° C., no more than about 85° C., no more than about 80° C., no more than about 75° C., no more than about 70° C., no more than about 65° C., no more than about 60° C., no more than about 55° C., no more than about 50° C., no more than about 45° C., no more than about 40° C., no more than about 35° C., no more than about 30° C., no more than about 25° C., no more than about 20° C., no more than about 15° C., or no more than about 10° C.

In another aspect, the present disclosure provides an imaging device configured for use in a medical procedure or a surgical procedure while the imaging device is within a body of an individual undergoing the medical procedure or the surgical procedure; wherein the imaging device comprises a set of thermal fins disposed on an exterior of the imaging device; and wherein the imaging device is configured to allow a maximum contact time with the individual undergoing the medical procedure or the surgical procedure that is at least about 2 times, at least about 3 times, at least about 4 times, at least about 5 times, at least about 6 times, at least about 7 times, at least about 8 times, at least about 9 times, at least about 10 times, at least about 11 times, at least about 12 times, at least about 13 times, at least about 14 times, at least about 15 times, at least about 16 times, at least about 17 times, at least about 18 times, at least about 19 times, or at least about 20 times, as compared to a maximum contact time with the individual undergoing the medical procedure or the surgical procedure of an imaging device not comprising the set of thermal fins.

In another aspect, the present disclosure provides a method for performing a medical procedure or a surgical procedure for an individual, comprising: (a) inserting at least a portion of an imaging device into a body of the individual; and (b) performing the medical procedure or the surgical procedure while the imaging device is within the body of the individual, wherein the imaging device comprises a coating on at least a portion of an exterior of the imaging device, and wherein the coating comprises a thermal emissivity of at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.97, at least about 0.98, or at least about 0.99.

In another aspect, the present disclosure provides a method for performing a medical procedure or a surgical procedure for an individual, comprising: (a) inserting at least a portion of an imaging device into a body of the individual; and (b) performing the medical procedure or the surgical procedure while the imaging device is within the body of the individual, wherein the imaging device comprises a coating on at least a portion of an exterior of the imaging device, and wherein the imaging device is configured to dissipate heat at a rate of at least about 2 times, at least about 3 times, at least about 4 times, at least about 5 times, at least about 6 times, at least about 7 times, at least about 8 times, at least about 9 times, at least about 10 times, at least about 11 times, at least about 12 times, at least about 13 times, at least about 14 times, at least about 15 times, at least about 16 times, at least about 17 times, at least about 18 times, at least about 19 times, or at least about 20 times, as compared to a rate of heat dissipation of an imaging device not comprising the coating.

In another aspect, the present disclosure provides a method for performing a medical procedure or a surgical procedure for an individual, comprising: (a) inserting at least a portion of an imaging device into a body of the individual; and (b) performing the medical procedure or the surgical procedure while the imaging device is within the body of the individual, wherein the imaging device comprises a coating on at least a portion of an exterior of the imaging device, and wherein the imaging device operates at a maximum temperature of no more than about 100° C., no more than about 95° C., no more than about 90° C., no more than about 85° C., no more than about 80° C., no more than about 75° C., no more than about 70° C., no more than about 65° C., no more than about 60° C., no more than about 55° C., no more than about 50° C., no more than about 45° C., no more than about 40° C., no more than about 35° C., no more than about 30° C., no more than about 25° C., no more than about 20° C., no more than about 15° C., or no more than about 10° C.

In some embodiments, the coating comprises a metal or metal alloy. In some embodiments, the coating comprises the metal. In some embodiments, the metal is an anodized metal. In some embodiments, the anodized metal is selected from the group consisting of aluminum, magnesium, and titanium. In some embodiments, the anodized metal is the aluminum. In some embodiments, the anodized metal is the magnesium. In some embodiments, the anodized metal is the titanium. In some embodiments, the coating comprises the metal alloy. In some embodiments, the metal alloy comprises a metal selected from the group consisting of aluminum, magnesium, and titanium. In some embodiments, the metal is the aluminum. In some embodiments, the metal is the magnesium. In some embodiments, the metal is the titanium. In some embodiments, the imaging device comprises an endoscope.

In another aspect, the present disclosure provides a method for performing a medical procedure or a surgical procedure for an individual, comprising: (a) inserting at least a portion of an imaging device into a body of the individual; and (b) performing the medical procedure or the surgical procedure while the imaging device is within the body of the individual, wherein the imaging device comprises a coating on at least a portion of an exterior of the imaging device, and wherein the imaging device is configured to allow a maximum contact time with the individual undergoing the medical procedure or the surgical procedure that is at least about 2 times, at least about 3 times, at least about 4 times, at least about 5 times, at least about 6 times, at least about 7 times, at least about 8 times, at least about 9 times, at least about 10 times, at least about 11 times, at least about 12 times, at least about 13 times, at least about 14 times, at least about 15 times, at least about 16 times, at least about 17 times, at least about 18 times, at least about 19 times, or at least about 20 times, as compared to a maximum contact time with the individual undergoing the medical procedure or the surgical procedure of an imaging device not comprising the coating.

In another aspect, the present disclosure provides a method for performing a medical procedure or a surgical procedure for an individual, comprising: (a) inserting at least a portion of an imaging device into a body of the individual; and (b) performing the medical procedure or the surgical procedure while the imaging device is within the body of the individual, wherein the imaging device comprises a set of thermal fins disposed on an exterior of the imaging device, and wherein the set of thermal fins increases a surface area of the exterior of the imaging device by a factor of at least about 2 times, at least about 3 times, at least about 4 times, at least about 5 times, at least about 6 times, at least about 7 times, at least about 8 times, at least about 9 times, at least about 10 times, at least about 11 times, at least about 12 times, at least about 13 times, at least about 14 times, at least about 15 times, at least about 16 times, at least about 17 times, at least about 18 times, at least about 19 times, or at least about 20 times, as compared to an imaging device not comprising the set of thermal fins.

In some embodiments, the set of thermal fins comprises a metal or metal alloy. In some embodiments, the set of thermal fins comprises the metal. In some embodiments, the metal is an anodized metal. In some embodiments, the anodized metal is selected from the group consisting of aluminum, magnesium, and titanium. In some embodiments, the anodized metal is the aluminum. In some embodiments, the anodized metal is the magnesium. In some embodiments, the anodized metal is the titanium. In some embodiments, the set of thermal fins comprises the metal alloy. In some embodiments, the metal alloy comprises a metal selected from the group consisting of aluminum, magnesium, and titanium. In some embodiments, the metal is the aluminum. In some embodiments, the metal is the magnesium. In some embodiments, the metal is the titanium. In some embodiments, the imaging device comprises an endoscope.

In another aspect, the present disclosure provides a method for performing a medical procedure or a surgical procedure for an individual, comprising: (a) inserting at least a portion of an imaging device into a body of the individual; and (b) performing the medical procedure or the surgical procedure while the imaging device is within the body of the individual, wherein the imaging device comprises a set of thermal fins disposed on an exterior of the imaging device, and wherein the imaging device is configured to dissipate heat at a rate of at least about 2 times, at least about 3 times, at least about 4 times, at least about 5 times, at least about 6 times, at least about 7 times, at least about 8 times, at least about 9 times, at least about 10 times, at least about 11 times, at least about 12 times, at least about 13 times, at least about 14 times, at least about 15 times, at least about 16 times, at least about 17 times, at least about 18 times, at least about 19 times, or at least about 20 times, as compared to a rate of heat dissipation of an imaging device not comprising the set of thermal fins.

In another aspect, the present disclosure provides a method for performing a medical procedure or a surgical procedure for an individual, comprising: (a) inserting at least a portion of an imaging device into a body of the individual; and (b) performing the medical procedure or the surgical procedure while the imaging device is within the body of the individual, wherein the imaging device comprises a set of thermal fins disposed on an exterior of the imaging device, and wherein the imaging device operates at a maximum temperature of no more than about 100° C., no more than about 95° C., no more than about 90° C., no more than about 85° C., no more than about 80° C., no more than about 75° C., no more than about 70° C., no more than about 65° C., no more than about 60° C., no more than about 55° C., no more than about 50° C., no more than about 45° C., no more than about 40° C., no more than about 35° C., no more than about 30° C., no more than about 25° C., no more than about 20° C., no more than about 15° C., or no more than about 10° C.

In another aspect, the present disclosure provides a method for performing a medical procedure or a surgical procedure for an individual, comprising: (a) inserting at least a portion of an imaging device into a body of the individual; and (b) performing the medical procedure or the surgical procedure while the imaging device is within the body of the individual, wherein the imaging device comprises a set of thermal fins disposed on an exterior of the imaging device, and wherein the imaging device is configured to allow a maximum contact time with the individual undergoing the medical procedure or the surgical procedure that is at least about 2 times, at least about 3 times, at least about 4 times, at least about 5 times, at least about 6 times, at least about 7 times, at least about 8 times, at least about 9 times, at least about 10 times, at least about 11 times, at least about 12 times, at least about 13 times, at least about 14 times, at least about 15 times, at least about 16 times, at least about 17 times, at least about 18 times, at least about 19 times, or at least about 20 times, as compared to a maximum contact time with the individual undergoing the medical procedure or the surgical procedure of an imaging device not comprising the set of thermal fins.

In another aspect, the present disclosure provides a method of increasing thermal performance of an imaging device configured for use in a medical procedure or a surgical procedure while the imaging device is within a body of an individual undergoing the medical procedure or the surgical procedure, wherein the method comprises disposing a coating onto at least a portion of an exterior of the imaging device, wherein the coating comprises a thermal emissivity of at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.97, at least about 0.98, or at least about 0.99.

In another aspect, the present disclosure provides a method of increasing thermal performance of an imaging device configured for use in a medical procedure or a surgical procedure while the imaging device is within a body of an individual undergoing the medical procedure or the surgical procedure, wherein the method comprises disposing a coating onto at least a portion of an exterior of the imaging device, thereby increasing a rate of heat dissipation of the imaging device by a factor of at least about 2 times, at least about 3 times, at least about 4 times, at least about 5 times, at least about 6 times, at least about 7 times, at least about 8 times, at least about 9 times, at least about 10 times, at least about 11 times, at least about 12 times, at least about 13 times, at least about 14 times, at least about 15 times, at least about 16 times, at least about 17 times, at least about 18 times, at least about 19 times, or at least about 20 times.

In another aspect, the present disclosure provides a method of increasing thermal performance of an imaging device configured for use in a medical procedure or a surgical procedure while the imaging device is within a body of an individual undergoing the medical procedure or the surgical procedure, wherein the method comprises disposing a coating onto at least a portion of an exterior of the imaging device, thereby increasing a rate of heat dissipation of the imaging device sufficient for the imaging device to operate at a maximum temperature of no more than about 100° C., no more than about 95° C., no more than about 90° C., no more than about 85° C., no more than about 80° C., no more than about 75° C., no more than about 70° C., no more than about 65° C., no more than about 60° C., no more than about 55° C., no more than about 50° C., no more than about 45° C., no more than about 40° C., no more than about 35° C., no more than about 30° C., no more than about 25° C., no more than about 20° C., no more than about 15° C., or no more than about 10° C.

In another aspect, the present disclosure provides a method of increasing thermal performance of an imaging device configured for use in a medical procedure or a surgical procedure while the imaging device is within a body of an individual undergoing the medical procedure or the surgical procedure, wherein the method comprises disposing a coating onto at least a portion of an exterior of the imaging device, thereby increasing a rate of heat dissipation of the imaging device sufficient for the imaging device to allow a maximum contact time with the individual undergoing the medical procedure or the surgical procedure that is increased by a factor of at least about 2 times, at least about 3 times, at least about 4 times, at least about 5 times, at least about 6 times, at least about 7 times, at least about 8 times, at least about 9 times, at least about 10 times, at least about 11 times, at least about 12 times, at least about 13 times, at least about 14 times, at least about 15 times, at least about 16 times, at least about 17 times, at least about 18 times, at least about 19 times, or at least about 20 times.

In another aspect, the present disclosure provides a method of increasing thermal performance of an imaging device configured for use in a medical procedure or a surgical procedure while the imaging device is within a body of an individual undergoing the medical procedure or the surgical procedure, wherein the method comprises disposing a set of thermal fins on an exterior of the imaging device, thereby increasing a surface area of the exterior of the imaging device by a factor of at least about 2 times, at least about 3 times, at least about 4 times, at least about 5 times, at least about 6 times, at least about 7 times, at least about 8 times, at least about 9 times, at least about 10 times, at least about 11 times, at least about 12 times, at least about 13 times, at least about 14 times, at least about 15 times, at least about 16 times, at least about 17 times, at least about 18 times, at least about 19 times, or at least about 20 times.

In some embodiments, the set of thermal fins comprises a metal or metal alloy. In some embodiments, the set of thermal fins comprises the metal. In some embodiments, the metal is an anodized metal. In some embodiments, the anodized metal is selected from the group consisting of aluminum, magnesium, and titanium. In some embodiments, the anodized metal is the aluminum. In some embodiments, the anodized metal is the magnesium. In some embodiments, the anodized metal is the titanium. In some embodiments, the set of thermal fins comprises the metal alloy. In some embodiments, the metal alloy comprises a metal selected from the group consisting of aluminum, magnesium, and titanium. In some embodiments, the metal is the aluminum. In some embodiments, the metal is the magnesium. In some embodiments, the metal is the titanium. In some embodiments, the imaging device comprises an endoscope.

In another aspect, the present disclosure provides a method of increasing thermal performance of an imaging device configured for use in a medical procedure or a surgical procedure while the imaging device is within a body of an individual undergoing the medical procedure or the surgical procedure, wherein the method comprises disposing a set of thermal fins on an exterior of the imaging device, thereby increasing a rate of heat dissipation of the imaging device by a factor of at least about 2 times, at least about 3 times, at least about 4 times, at least about 5 times, at least about 6 times, at least about 7 times, at least about 8 times, at least about 9 times, at least about 10 times, at least about 11 times, at least about 12 times, at least about 13 times, at least about 14 times, at least about 15 times, at least about 16 times, at least about 17 times, at least about 18 times, at least about 19 times, or at least about 20 times.

In another aspect, the present disclosure provides a method of increasing thermal performance of an imaging device configured for use in a medical procedure or a surgical procedure while the imaging device is within a body of an individual undergoing the medical procedure or the surgical procedure, wherein the method comprises disposing a set of thermal fins on an exterior of the imaging device, thereby increasing a rate of heat dissipation of the imaging device sufficient for the imaging device to operate at a maximum temperature of no more than about 100° C., no more than about 95° C., no more than about 90° C., no more than about 85° C., no more than about 80° C., no more than about 75° C., no more than about 70° C., no more than about 65° C., no more than about 60° C., no more than about 55° C., no more than about 50° C., no more than about 45° C., no more than about 40° C., no more than about 35° C., no more than about 30° C., no more than about 25° C., no more than about 20° C., no more than about 15° C., or no more than about 10° C.

In another aspect, the present disclosure provides a method of increasing thermal performance of an imaging device configured for use in a medical procedure or a surgical procedure while the imaging device is within a body of an individual undergoing the medical procedure or the surgical procedure, wherein the method comprises disposing a set of thermal fins on an exterior of the imaging device, thereby increasing a rate of heat dissipation of the imaging device sufficient for the imaging device to allow a maximum contact time with the individual undergoing the medical procedure or the surgical procedure that is increased by a factor of at least about 2 times, at least about 3 times, at least about 4 times, at least about 5 times, at least about 6 times, at least about 7 times, at least about 8 times, at least about 9 times, at least about 10 times, at least about 11 times, at least about 12 times, at least about 13 times, at least about 14 times, at least about 15 times, at least about 16 times, at least about 17 times, at least about 18 times, at least about 19 times, or at least about 20 times.

In some embodiments, systems and methods of the present disclosure may leverage a high emissivity coating and/or a set of thermal fins to improve thermal performance of endoscopes.

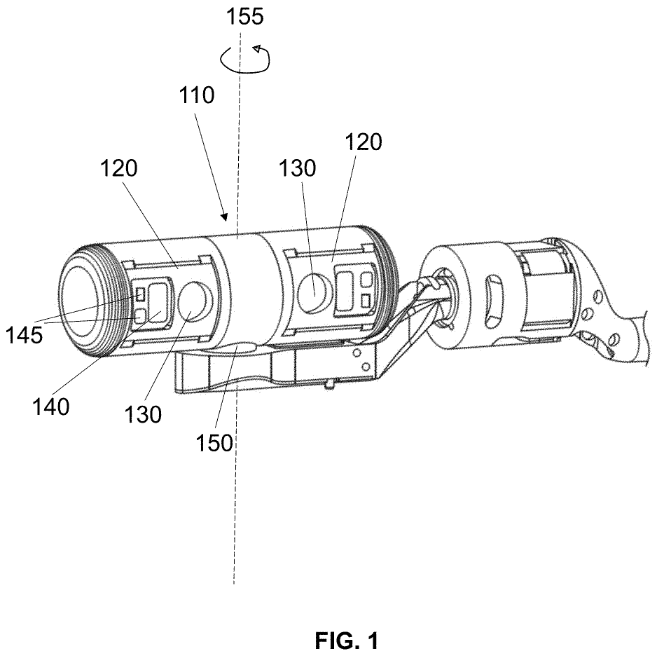

For example, FIG. 1 shows an example of two endoscopes, including a top endoscope having a thermal coating and a bottom endoscope not having a thermal coating. shows an imaging device 110 comprising a lens 130 behind a window 120 in an orthogonal direction to insertion. The imaging device 110 may rotate at a point of rotation 150 about an axis of rotation 155 to a desired orientation. In some embodiments, translation towards a second window or lens may not be needed if two wiping objects were to be used. In some embodiments, the imaging device 110 may comprise a light source comprising a light emitting diode (LED) 140 and/or one or more sensors 145 behind a window 120. The high emissivity thermal coating may be achieved via the anodization process of aluminum, which may be used extensively in various industries to protect components against corrosion and scratches, increase durability of components, and to change aesthetics. In this case, anodization may be used to produce a coating comprising a metal or metal alloy with a high thermal emissivity. By coating a surface of an endoscope with such a high thermal emissivity coating, the black body emissivity coefficient of the surface of the endoscope is effectively increased from about 0.05 for the uncoated aluminum to about 0.8 for the anodized aluminum. Alternatively, the coating may comprise another metal (e.g., magnesium or titanium) or a metal alloy (e.g., containing aluminum, magnesium, or titanium) or a polymer (e.g., PTFE) or a plastic (e.g., PEEK, glazed porcelain, polypropylene).

This increase in the emissivity coefficient may enable the endoscope to transfer heat from the endoscope to the environment through radiation at an accelerated rate, thereby resulting in accelerated heat dissipation and producing a lower surface temperature for the endoscope during operation. The resulting lower surface temperature of the endoscope reduces the risks to the patient due to tissue burns, if the endoscope comes into contact with the patient.

The high emissivity coating may be achieved by anodizing the aluminum casing of the camera, which allows the camera surface to transfer heat out of the camera's electronics through radiation up to 15 times faster than without the coating. The coating may not affect the camera's design, other than changing the color of the surface from aluminum gray to black. As a result of the coating, the camera may be able to transfer heat out of the endoscope, which results in a decreased surface temperature during normal camera operation. The lower surface temperature results in a decreased chance of burning the patient and an increase in allowable contact time if the camera accidently comes into contact with tissue.

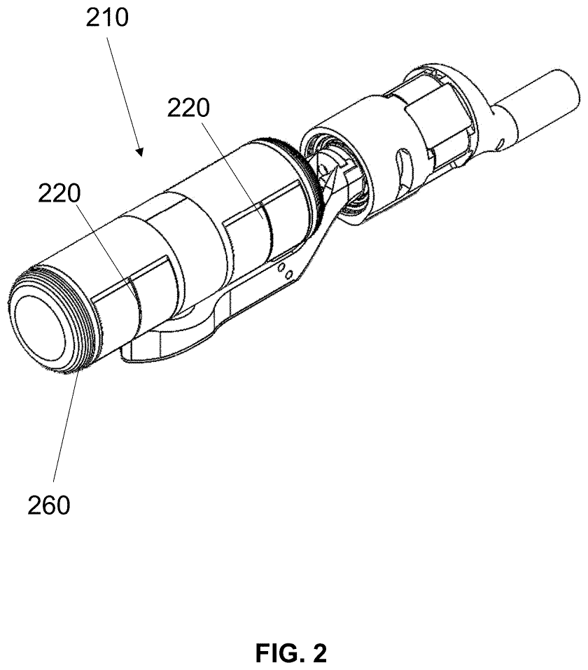

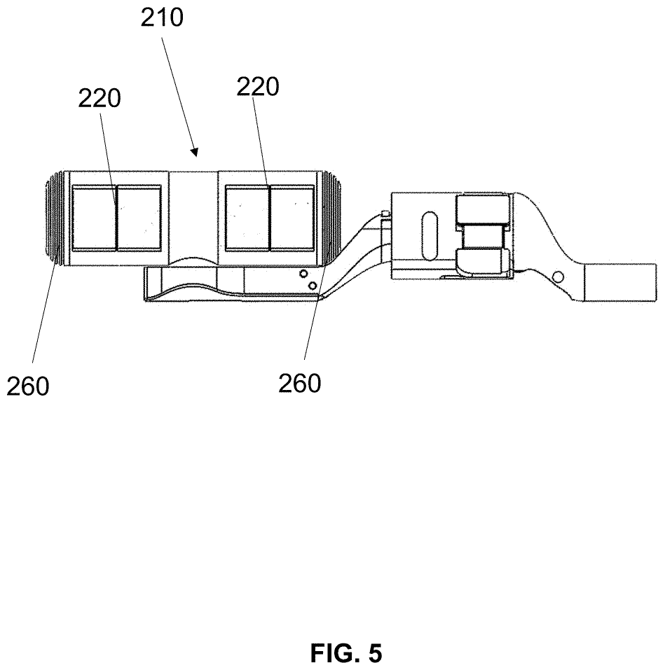

In some embodiments, a set of thermal fins may be used as heat sinks implemented in a micro design on endoscopes to reduce temperature values and surface area contact for overall reduction in risk of burning the patient with long-term contact. FIG. 2 and FIG. 5 show a perspective view and a front view, respectively, of an example of a camera 210 having window 220 and thermal fins 260 visible on the extremes of the camera. Thermal fins (or heat fins) can help reduce the maximum temperature of an endoscope that would otherwise reach high temperatures. In the case of accidental contact of the light emission section of the endoscope with the patient, the addition of fins will increase the time of allowable contact before damaging patient tissue. In some embodiments, the plurality of fins comprises at least 1, 2, 3, 4, 5, 6, 7 ,8, 9, 10, 20, 30, 40, 50, 60, 70, 80 , 90, or 100 thermal fins.

In some embodiments, the thermal fins may be mechanical fins disposed on the surface of the camera body. These fins may be similar to those of a heatsink, which are designed to be smooth (not sharp) to the skin and coated with hydrophobic and/or oleophobic substances to reduce potential build-up of liquids on the surface that may potentially interfere with heat transfer. When coming in contact with skin, the temperature of the fins may be lower than that of the original endoscope device, due to the added surface area from the fins. The surface area of any potential contact may also be smaller, thereby reducing the amount of thermal conduction to the portion of the individual's body that is contacted with the endoscope. Further, the fins may be designed to be black in color, with a high thermal emissivity (e.g., by using a coating, as described herein), to increase radiative heat emission from the surface.

In some embodiments, the thermal fins are designed from a less thermally conductive material, such that the temperature at the end of the fins is substantially lower than the beginning of the fins. This may reduce contact area and thermal temperature at the point of contact (e.g., between the endoscope and the anatomy of the individual's body).

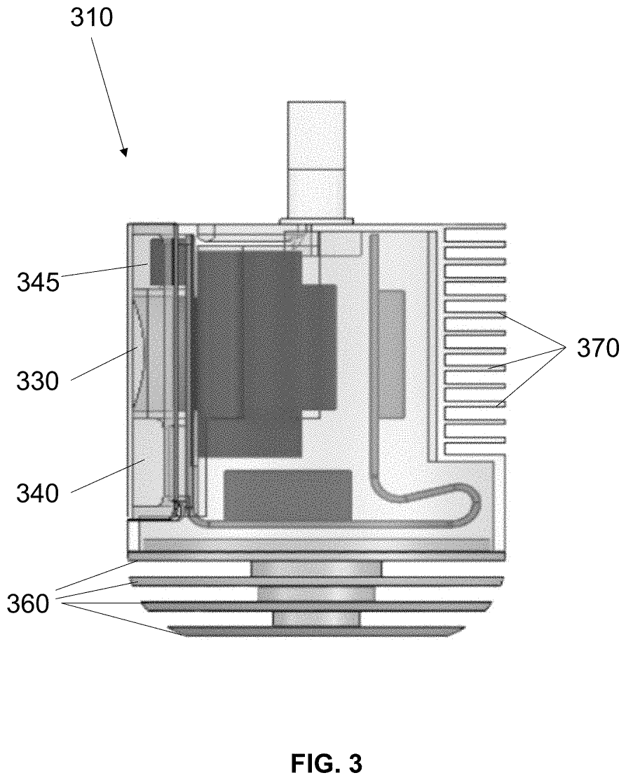

FIG. 3 shows an example of thermal fins 360, 370 of an endoscope 310. For example, two sections of vertical fins 370 and radial fins are used to maximize surface area of the module. In some embodiments, the thermal fins may comprise cylindrical fins 360, which may be disposed at the ends of the endoscope to allow for a very large cross section of surface area and heat dissipation. For example, this may be adapted easily for an endoscope where the wires transfer down the center portion. In some embodiments, the imaging device 310 comprising a lens 330, a light source comprising a light emitting diode (LED) 340 and/or one or more sensors 345 behind a window.

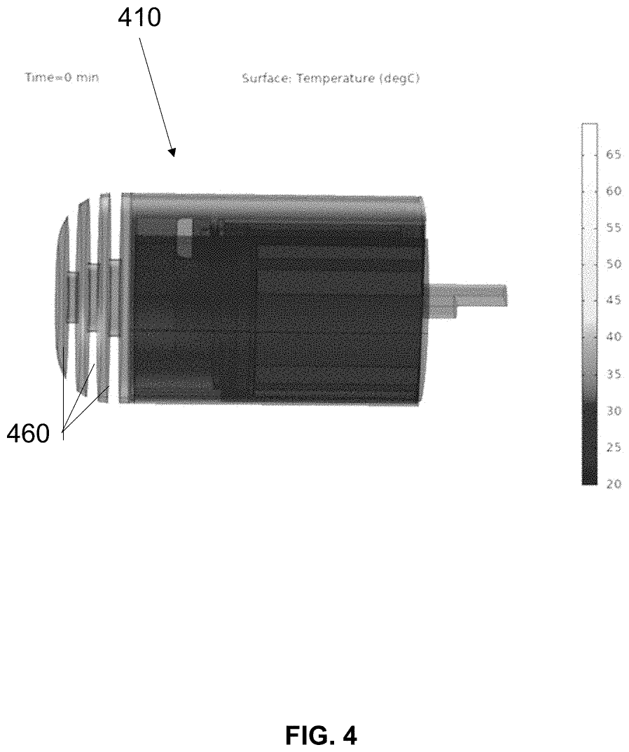

FIG. 4 shows an example thermal fins 460 of an endoscope 410 where the linear slots of the thermal fins 460 allow for reduced temperature at the contact surfaces but not substantially increased overall temperature. In some embodiments, the thermal fins for the endoscope may comprise one or more of: vertical fins, radial fins, cylindrical fins, and linear slots.

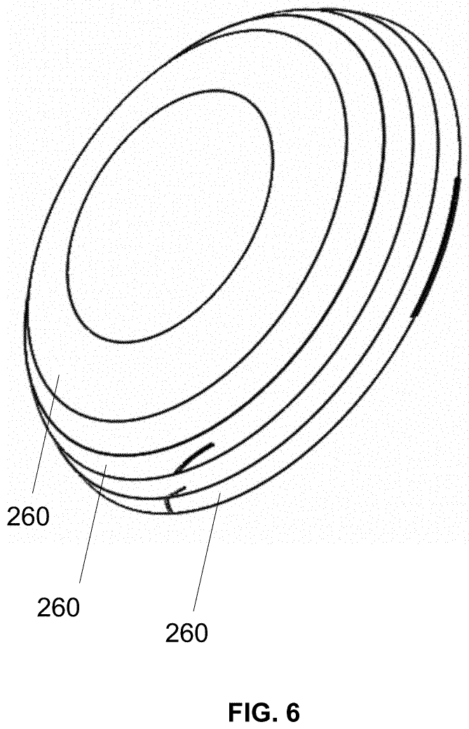

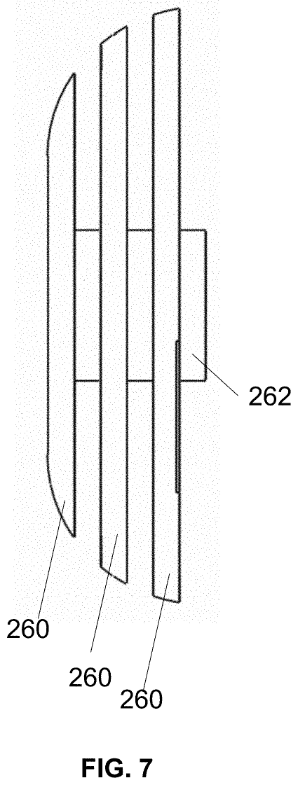

FIGS. 6 and 7 show a perspective view and a side view, respectively, of exemplary embodiments of a plurality of cylindrical thermal fins 260. In some embodiments, the cylindrical thermal fins may be attached to the imaging device by a connector 262. In some embodiments, the cylindrical thermal fins may be disposed at the ends of the endoscope to allow for a larger cross section of surface area and heat dissipation. In some embodiments, the plurality of thermal fins comprises 1, 2, 3, 4, 5, 6, 7 ,8, 9, or 10 cylindrical thermal fins. In some embodiments, the plurality of thermal fins comprises 3 cylindrical thermal fins.

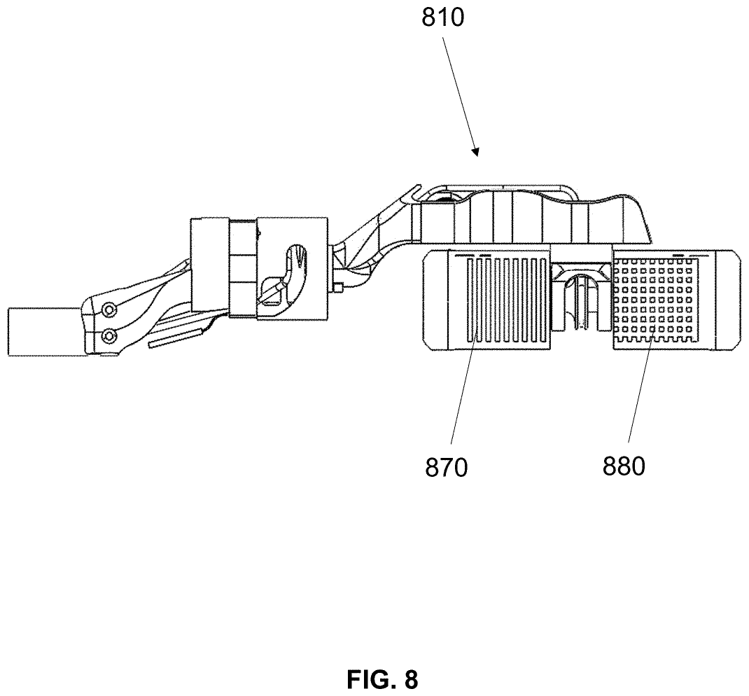

FIG. 8 shows an exemplary embodiment of an isometric view of the imaging device 810 comprising a plurality of thermal fins 870, 880. FIG. 8 shows an exemplary embodiment having a plurality of elongated thermal fins, also referred herein as vertical thermal fins, 870 on the left side of the imaging device and a plurality of thermal fins in a grid pattern 880 on the right side of the imaging device. Notably, the thermals fins may be the same or different on each side of the imaging device.

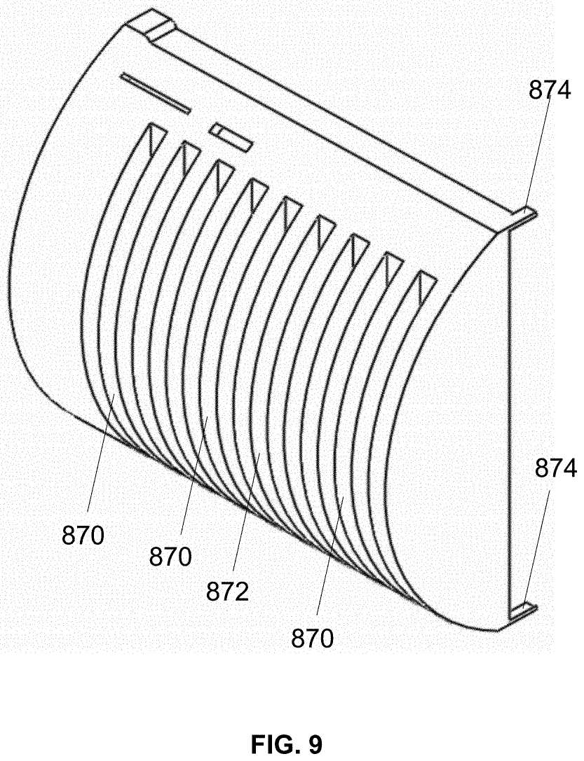

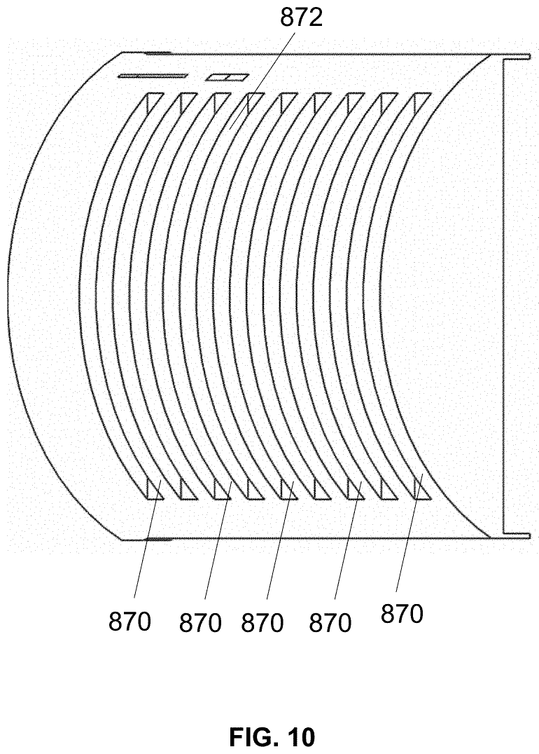

FIGS. 9 and 10 show a perspective view and a front view, respectively, of exemplary embodiments of a plurality of vertical thermal fins 870 separated by a plurality of linear slots 872. In some embodiments, the cylindrical thermal fins may be attached to the imaging device by one or more connectors 874. In some embodiments, the vertical thermal fins may be disposed on a backside of the imaging device, or a side opposite the lens of the imaging device, to allow for imaging from the front side of the imaging device. In some embodiments, the placement of the vertical thermal fins allows for faster dissipation of heat generated from the light source and other components of the imaging device from the proximity of the thermal fins to the components of the imaging that generate heat. In some embodiments, the plurality of thermal fins comprises at least 1, 2, 3, 4, 5, 6, 7, 8, 9, 10, 15, 20, 25, 30, 35, 40, 45, or 50 vertical thermal fins. In some embodiments, the plurality of thermal fins comprises 8 vertical fins.

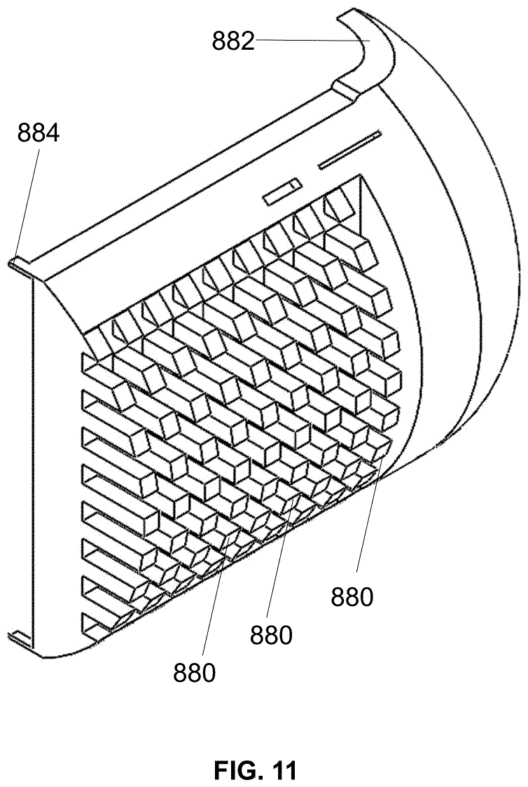

FIG. 11 shows a perspective view of exemplary embodiments of a plurality of thermal fins in a grid pattern 880. In some embodiments, the plurality of thermal fins in a grid pattern are pin-shaped. In some embodiments, the thermal fins may be attached to the imaging device by one or more connectors 882, 884. In some embodiments, the thermal fins may be disposed on a backside of the imaging device, or a side opposite the lens of the imaging device, to allow for imaging from the front side of the imaging device. In some embodiments, the placement of the thermal fins allows for faster dissipation of heat generated from the light source and other components of the imaging device from the proximity of the thermal fins to the components of the imaging that generate heat. In some embodiments, the plurality of thermal fins comprises at least 5, 10, 20, 30, 40, 50, 60, 70, 80, 90, or 100 thermal fins in a grid pattern. In some embodiments, the plurality of thermal fins comprises 1 to 100, 10 to 100, 30 to 100, or 50 to 100 thermal fins in a grid pattern.

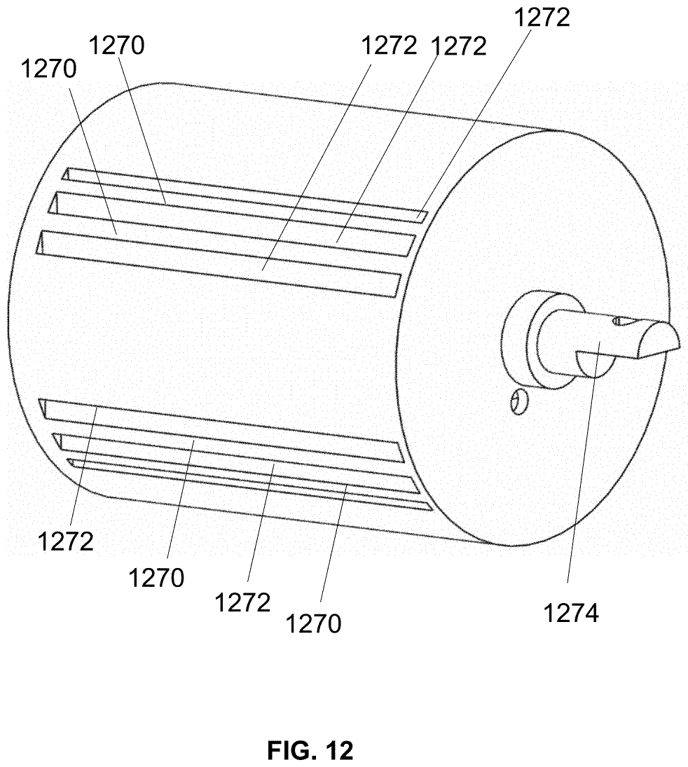

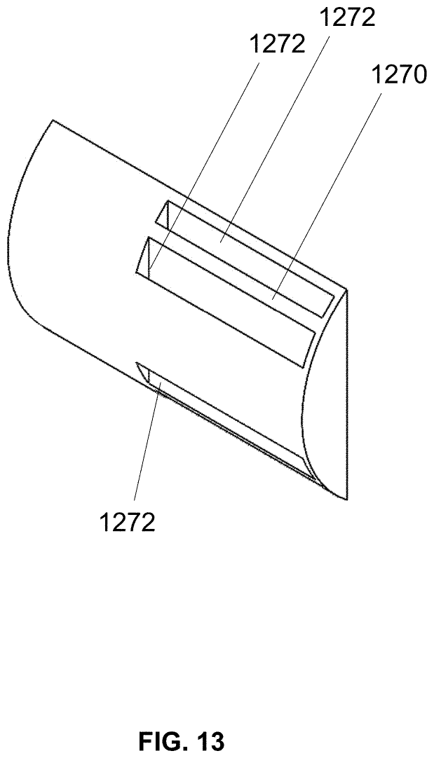

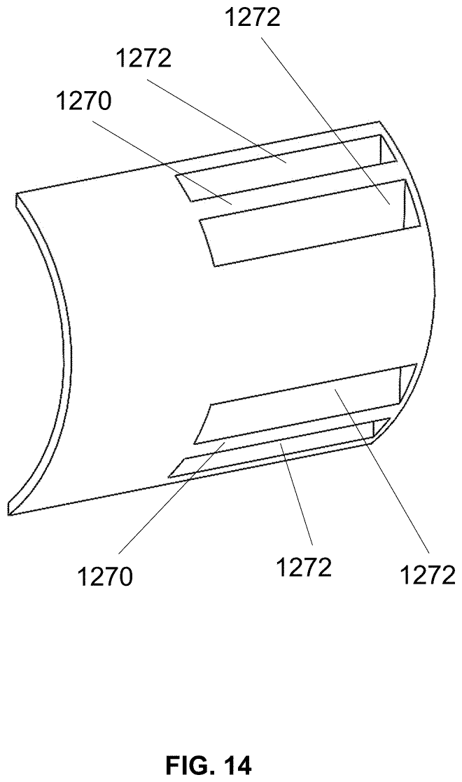

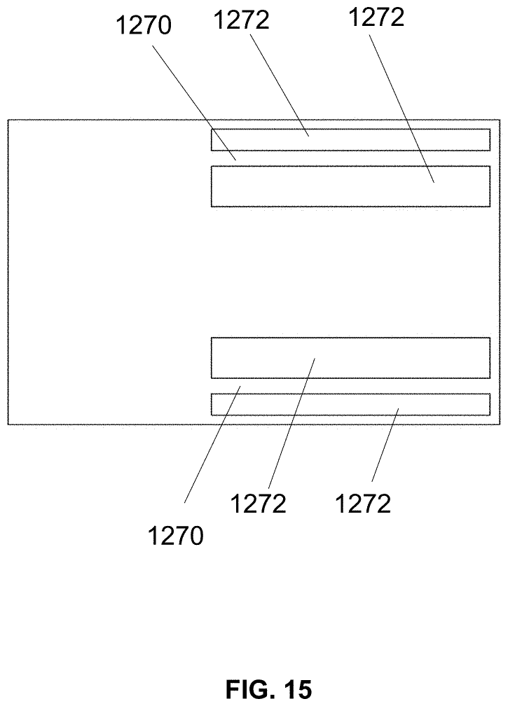

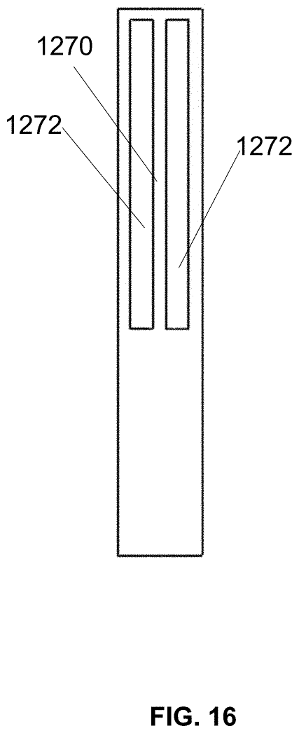

FIG. 12 shows a perspective view of exemplary embodiments of a plurality of thermal fins 1270 separated by a plurality of linear slots 1272. Notably, the linear slots 1272 may each have different dimensions such as width or may be substantially similar in dimension. In some embodiments, the thermal fins may be attached to the imaging device by one or more connectors 1274. FIGS. 13, 14, 15, and 16 show a right perspective view, a left perspective view, a front view, and a side view of the plurality of thermal fins 1270 separated by the plurality of linear slots 1272. In some embodiments, the thermal fins may be placed on a backside of the imaging device, or a side opposite the lens of the imaging device, to allow for imaging from the front side of the imaging device. In some embodiments, the placement of the thermal fins allows for faster dissipation of heat generated from the light source and other components of the imaging device from the proximity of the thermal fins to the components of the imaging that generate heat. In some embodiments, the plurality of thermal fins comprises at least 1, 2, 3, 4, 5, 6, 7, 8, 9, or 10 thermal fins separated by linear slots. In some embodiments, the plurality of thermal fins comprises 1 to 10 or 1 to 5 thermal fins separated by linear slots.

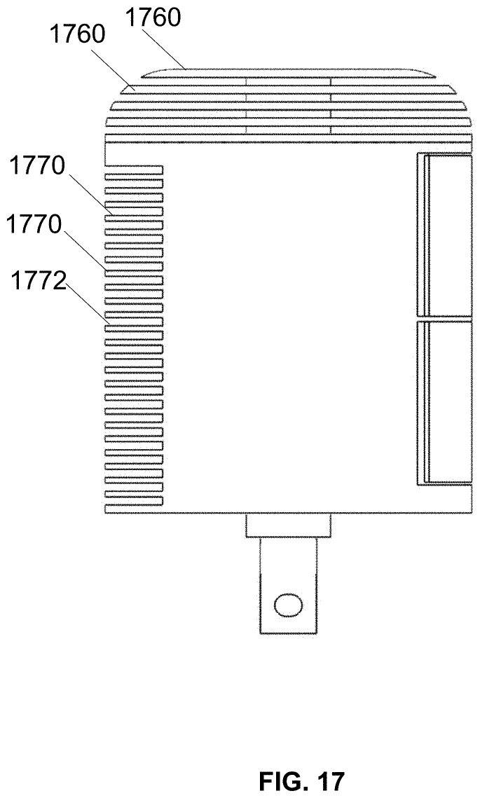

FIG. 17 shows a side view of plurality of thermal fins, including a plurality of cylindrical thermal fins 1760 and a plurality of thermal fins 1770 separated by the plurality of linear slots 1772.

### EXEMPLARY EMBODIMENTS

Among the exemplary embodiments are:

Embodiment 1 comprises an imaging device configured for use in a medical procedure or a surgical procedure within a body of an individual; wherein the imaging device comprises a coating on at least a portion of an exterior of the imaging device; and wherein the coating comprises a thermal emissivity of about 0.20 to 1. Embodiment 2 comprises an imaging device configured for use in a medical procedure or a surgical procedure within a body of an individual; wherein the imaging device comprises a coating on at least a portion of an exterior of the imaging device; and wherein the imaging device is configured to dissipate heat at a rate of about 2 to 20 times as compared to a rate of heat dissipation of an imaging device not comprising the coating. Embodiment 3 comprises an imaging device configured for use in a medical procedure or a surgical procedure within a body of an individual; wherein the imaging device comprises a coating on at least a portion of an exterior of the imaging device; and wherein the imaging device operates at a maximum temperature of about 10° C. to 100° C. Embodiment 4 comprises an imaging device configured for use in a medical procedure or a surgical procedure within a body of an individual; wherein the imaging device comprises a coating on at least a portion of an exterior of the imaging device; and wherein the imaging device is configured to allow a maximum contact time with the individual undergoing the medical procedure or the surgical procedure that is about 2 to 20 times as compared to a maximum contact time with the individual undergoing the medical procedure or the surgical procedure of an imaging device not comprising the coating. Embodiment 5 comprises the imaging device of any one of embodiments 1 to 4, wherein the coating comprises a metal or metal alloy. Embodiment 6 comprises the imaging device of embodiment 5, wherein the coating comprises the metal. Embodiment 7 comprises the imaging device of embodiment 7, wherein the metal is selected from the group consisting of aluminum, magnesium, and titanium. Embodiment 8 comprises the imaging device of any one of embodiments 1 to 4, wherein the coating comprises polytetrafluoroethylene (PTFE). Embodiment 9 comprises the imaging device of any one of embodiments 1 to 8, wherein the coating comprises more than one layer. Embodiment 10 comprises the imaging device of embodiment 9, wherein the more than one layer comprises an inner layer comprising a conductive material. Embodiment 11 comprises the imaging device of embodiment 10, wherein the conductive material comprises aluminum or copper or a combination thereof. Embodiment 12 comprises the imaging device of embodiment 9 or 11, wherein the more than one layer comprises an outer layer comprising a material with a higher thermal emissivity than the inner layer. Embodiment 13 comprises the imaging device of embodiment 12, wherein the material with a higher thermal emissivity comprises PEEK, glazed porcelain, or polypropylene or a combination thereof.

Embodiment 14 comprises an imaging device configured for use in a medical procedure or a surgical procedure within a body of an individual; wherein the imaging device comprises a set of thermal fins disposed on an exterior of the imaging device; and wherein the set of thermal fins increases a surface area of the exterior of the imaging device by a factor of about 2 to 20 times as compared to an imaging device not comprising the set of thermal fins. Embodiment 15 comprises an imaging device configured for use in a medical procedure or a surgical procedure within a body of an individual; wherein the imaging device comprises a set of thermal fins disposed on an exterior of the imaging device; and wherein the imaging device is configured to dissipate heat at a rate of about 2 to 20 times as compared to a rate of heat dissipation of an imaging device not comprising the set of thermal fins. Embodiment 16 comprises an imaging device configured for use in a medical procedure or a surgical procedure while the imaging device is within a body of an individual undergoing the medical procedure or the surgical procedure; wherein the imaging device comprises a set of thermal fins disposed on an exterior of the imaging device; and wherein the imaging device operates at a maximum temperature of about 10° C. to 100° C. Embodiment 17 comprises an imaging device configured for use in a medical procedure or a surgical procedure within a body of an individual; wherein the imaging device comprises a set of thermal fins disposed on an exterior of the imaging device; and wherein the imaging device is configured to allow a maximum contact time with the individual undergoing the medical procedure or the surgical procedure that is about 2 to 20 times as compared to a maximum contact time with the individual undergoing the medical procedure or the surgical procedure of an imaging device not comprising the set of thermal fins. Embodiment 18 comprises the imaging device of any one of embodiments 14 to 17, wherein the imaging device comprises a metal or metal alloy. Embodiment 19 comprises the imaging device of embodiment 18,wherein the imaging device comprises the metal. Embodiment 20 comprises the imaging device of embodiment 19, wherein the metal is selected from the group consisting of aluminum, magnesium, and titanium. Embodiment 21 comprises the imaging device of any one of claims 1 to 20, wherein the imaging device comprises an endoscope. Embodiment 22 comprises the imaging device of any one of claims 14 to 17, wherein the imaging device comprises a coating comprising polytetrafluoroethylene (PTFE). Embodiment 23 comprises the imaging device of any one of claims 14 to 22, wherein the imaging device comprises a coating comprising more than one layer. Embodiment 24 comprises the imaging device of claim 23, wherein the more than one layer comprises an inner layer comprising a conductive material. Embodiment 25 comprises the imaging device of claim 24, wherein the conductive material comprises aluminum or copper or a combination thereof. Embodiment 26 comprises the imaging device of any one of claims 23 to 25, wherein the more than one layer comprises an outer layer comprising a material with a higher thermal emissivity than the inner layer. Embodiment 27 comprises the imaging device of claim 26, wherein the material with a higher thermal emissivity comprises PEEK, glazed porcelain, or polypropylene or a combination thereof. Embodiment 28 comprises the imaging device of any one of claims 14 to 27, wherein the thermal fins comprise one or more of: vertical fins, radial fins, cylindrical fins, linear slots, or pin fins. Embodiment 29 comprises the imaging device of claim 28, wherein the pin fins are arranged in a grid pattern. Embodiment 30 comprises the imaging device of any one of claims 14 to 29, wherein the thermal fins comprise at least 1, 2, 3, 4, 5, 6, 7, 8, 9, or 10 thermal fins.

Embodiment 31 comprises a method for performing a medical procedure or a surgical procedure for an individual, comprising: inserting at least a portion of an imaging device into a body of the individual; and performing the medical procedure or the surgical procedure while the imaging device is within the body of the individual, wherein the imaging device comprises a coating on at least a portion of an exterior of the imaging device, and wherein the coating comprises a thermal emissivity of about 0.20 to 0.99. Embodiment 32 comprises a method for performing a medical procedure or a surgical procedure for an individual, comprising: inserting at least a portion of an imaging device into a body of the individual; and performing the medical procedure or the surgical procedure while the imaging device is within the body of the individual, wherein the imaging device comprises a coating on at least a portion of an exterior of the imaging device, and wherein the imaging device is configured to dissipate heat at a rate of about 2 to 20 times as compared to a rate of heat dissipation of an imaging device not comprising the coating. Embodiment 33 comprises a method for performing a medical procedure or a surgical procedure for an individual, comprising: inserting at least a portion of an imaging device into a body of the individual; and performing the medical procedure or the surgical procedure while the imaging device is within the body of the individual, wherein the imaging device comprises a coating on at least a portion of an exterior of the imaging device, and wherein the imaging device operates at a maximum temperature of about 10° C. to 100° C. Embodiment 34 comprises a method for performing a medical procedure or a surgical procedure for an individual, comprising: inserting at least a portion of an imaging device into a body of the individual; and performing the medical procedure or the surgical procedure while the imaging device is within the body of the individual, wherein the imaging device comprises a coating on at least a portion of an exterior of the imaging device, and wherein the imaging device is configured to allow a maximum contact time with the individual undergoing the medical procedure or the surgical procedure that is about 2 to 20 times as compared to a maximum contact time with the individual undergoing the medical procedure or the surgical procedure of an imaging device not comprising the coating. Embodiment 35 comprises the method of any one of claims 31 to 34, wherein the coating comprises a metal or metal alloy. Embodiment 36 comprises the method of claim 35, wherein the coating comprises the metal. Embodiment 37 comprises the method of claim 36, wherein the metal is selected from the group consisting of aluminum, magnesium, and titanium. Embodiment 38 comprises the method of any one of claims 31 to 34, wherein the coating comprises polytetrafluoroethylene (PTFE). Embodiment 39 comprises the method of any one of claims 31 to 38, wherein the coating comprises more than one layer. Embodiment 40 comprises the method of claim 39, wherein the more than one layer comprises an inner layer comprising a conductive material. Embodiment 41 comprises the method of claim 40, wherein the conductive material comprises aluminum or copper or a combination thereof. Embodiment 42 comprises the method of any one of claims 39 to 41, wherein the more than one layer comprises an outer layer comprising a material with a higher thermal emissivity than the inner layer. Embodiment 43 comprises the method of claim 42, wherein the material with a higher thermal emissivity comprises PEEK, glazed porcelain, or polypropylene or a combination thereof

Embodiment 44 comprises a method for performing a medical procedure or a surgical procedure for an individual, comprising: inserting at least a portion of an imaging device into a body of the individual; and performing the medical procedure or the surgical procedure while the imaging device is within the body of the individual, wherein the imaging device comprises a set of thermal fins disposed on an exterior of the imaging device, and wherein the set of thermal fins increases a surface area of the exterior of the imaging device by a factor of about 2 to 20 times as compared to an imaging device not comprising the set of thermal fins. Embodiment 45 comprises a method for performing a medical procedure or a surgical procedure for an individual, comprising: inserting at least a portion of an imaging device into a body of the individual; and performing the medical procedure or the surgical procedure while the imaging device is within the body of the individual, wherein the imaging device comprises a set of thermal fins disposed on an exterior of the imaging device, and wherein the imaging device is configured to dissipate heat at a rate of about 2 to 20 times as compared to a rate of heat dissipation of an imaging device not comprising the set of thermal fins. Embodiment 46 comprises a method for performing a medical procedure or a surgical procedure for an individual, comprising: inserting at least a portion of an imaging device into a body of the individual; and performing the medical procedure or the surgical procedure while the imaging device is within the body of the individual, wherein the imaging device comprises a set of thermal fins disposed on an exterior of the imaging device, and wherein the imaging device operates at a maximum temperature of about 10° C. to 100° C. Embodiment 47 comprises a method for performing a medical procedure or a surgical procedure for an individual, comprising: inserting at least a portion of an imaging device into a body of the individual; and performing the medical procedure or the surgical procedure while the imaging device is within the body of the individual, wherein the imaging device comprises a set of thermal fins disposed on an exterior of the imaging device, and wherein the imaging device is configured to allow a maximum contact time with the individual undergoing the medical procedure or the surgical procedure that is about 2 to 20 times as compared to a maximum contact time with the individual undergoing the medical procedure or the surgical procedure of an imaging device not comprising the set of thermal fins. Embodiment 48 comprises the method of any one of claims 44 to 47, wherein the imaging device comprises a metal or metal alloy. Embodiment 49 comprises the method of claim 48, wherein the imaging device comprises the metal. Embodiment 50 comprises the method of claim 49, wherein the metal is selected from the group consisting of aluminum, magnesium, and titanium. Embodiment 51 comprises the method of any one of claims 31 to 50, wherein the imaging device comprises an endoscope. Embodiment 52 comprises the method of any one of claims 44 to 47, wherein the imaging device comprises a coating comprising polytetrafluoroethylene (PTFE). Embodiment 53 comprises the method of any one of claims 44 to 52, wherein the imaging device comprises a coating comprising more than one layer. Embodiment 54 comprises the method of claim 53, wherein the more than one layer comprises an inner layer comprising a conductive material. Embodiment 55 comprises the method of claim 54, wherein the conductive material comprises aluminum or copper or a combination thereof. Embodiment 56 comprises the method of any one of claims 53 to 55, wherein the more than one layer comprises an outer layer comprising a material with a higher thermal emissivity than the inner layer. Embodiment 57 comprises the method of claim 56, wherein the material with a higher thermal emissivity comprises PEEK, glazed porcelain, or polypropylene or a combination thereof. Embodiment 58 comprises the method of any one of claims 44 to 57, wherein the thermal fins comprise one or more of: vertical fins, radial fins, cylindrical fins, linear slots, or pin fins. Embodiment 59 comprises the method of claim 59, wherein the pin fins are arranged in a grid pattern. Embodiment 60 comprises the method of any one of claims 44 to 59, wherein the thermal fins comprise at least 1, 2, 3, 4, 5, 6, 7, 8, 9, or 10 thermal fins.

Embodiment 61 comprises a method of increasing thermal performance of an imaging device configured for use in a medical procedure or a surgical procedure while the imaging device is within a body of an individual undergoing the medical procedure or the surgical procedure, wherein the method comprises disposing a coating onto at least a portion of an exterior of the imaging device, wherein the coating comprises a thermal emissivity of about 0.20 to 0.99. Embodiment 62 comprises a method of increasing thermal performance of an imaging device configured for use in a medical procedure or a surgical procedure while the imaging device is within a body of an individual undergoing the medical procedure or the surgical procedure, wherein the method comprises disposing a coating onto at least a portion of an exterior of the imaging device, thereby increasing a rate of heat dissipation of the imaging device by a factor of about 2 to 20 times. Embodiment 63 comprises a method of increasing thermal performance of an imaging device configured for use in a medical procedure or a surgical procedure while the imaging device is within a body of an individual undergoing the medical procedure or the surgical procedure, wherein the method comprises disposing a coating onto at least a portion of an exterior of the imaging device, thereby increasing a rate of heat dissipation of the imaging device sufficient for the imaging device to operate at a maximum temperature of about 10° C. to 100° C. Embodiment 64 comprises a method of increasing thermal performance of an imaging device configured for use in a medical procedure or a surgical procedure while the imaging device is within a body of an individual undergoing the medical procedure or the surgical procedure, wherein the method comprises disposing a coating onto at least a portion of an exterior of the imaging device, thereby increasing a rate of heat dissipation of the imaging device sufficient for the imaging device to allow a maximum contact time with the individual undergoing the medical procedure or the surgical procedure that is increased by a factor of about 2 to 20 times. Embodiment 65 comprises the method of any one of claims 61 to 64, wherein the coating comprises a metal or metal alloy. Embodiment 66 comprises the method of claim 65, wherein the coating comprises the metal. Embodiment 67 comprises the method of claim 66, wherein the metal is selected from the group consisting of aluminum, magnesium, and titanium. Embodiment 68 comprises the method of any one of claims 61 to 64, wherein the coating comprises polytetrafluoroethylene (PTFE). Embodiment 69 comprises the method of any one of claims 61 to 68, wherein the coating comprises more than one layer. Embodiment 70 comprises the method of claim 69, wherein the more than one layer comprises an inner layer comprising a conductive material. Embodiment 71 comprises the method of claim 70, wherein the conductive material comprises aluminum or copper or a combination thereof. Embodiment 72 comprises the method of any one of claims 69 to 71, wherein the more than one layer comprises an outer layer comprising a material with a higher thermal emissivity than the inner layer. Embodiment 73 comprises the method of claim 72, wherein the material with a higher thermal emissivity comprises PEEK, glazed porcelain, or polypropylene or a combination thereof. Embodiment 74 comprises a method of increasing thermal performance of an imaging device during a medical procedure or a surgical procedure within a body of an individual, wherein the method comprises disposing a set of thermal fins on an exterior of the imaging device, thereby increasing a surface area of the exterior of the imaging device by a factor of about 2 to 20 times. Embodiment 75 comprises a method of increasing thermal performance of an imaging device during a medical procedure or a surgical procedure within a body of an individual, wherein the method comprises disposing a set of thermal fins on an exterior of the imaging device, thereby increasing a rate of heat dissipation of the imaging device by a factor of about 2 to 20 times. Embodiment 76 comprises a method of increasing thermal performance of an imaging device during a medical procedure or a surgical procedure within a body of an individual, wherein the method comprises disposing a set of thermal fins on an exterior of the imaging device, thereby increasing a rate of heat dissipation of the imaging device sufficient for the imaging device to operate at a maximum temperature of about 10° C. to 100° C. Embodiment 77 comprises a method of increasing thermal performance of an imaging device during a medical procedure or a surgical procedure within a body of an individual, wherein the method comprises disposing a set of thermal fins on an exterior of the imaging device, thereby increasing a rate of heat dissipation of the imaging device sufficient for the imaging device to allow a maximum contact time with the individual undergoing the medical procedure or the surgical procedure that is increased by a factor of about 2 to 20 times. Embodiment 78 comprises the method of any one of claims 74 to 77, wherein the imaging device comprises a metal or metal alloy. Embodiment 79 comprises the method of claim 78, wherein the imaging device comprises the metal. Embodiment 80 comprises the method of claim 79, wherein the metal is selected from the group consisting of aluminum, magnesium, and titanium. Embodiment 81 comprises the method of any one of claims 74 to 7778, wherein the imaging device comprises a coating comprising polytetrafluoroethylene (PTFE). Embodiment 82 comprises the method of any one of claims 74 to 81, wherein the imaging device comprises a coating comprising more than one layer. Embodiment 83 comprises the method of claim 82, wherein the more than one layer comprises an inner layer comprising a conductive material. Embodiment 84 comprises the method of claim 83, wherein the conductive material comprises aluminum or copper or a combination thereof. Embodiment 85 comprises the method of any one of claims 82 to 84, wherein the more than one layer comprises an outer layer comprising a material with a higher thermal emissivity than the inner layer. Embodiment 86 comprises the method of claim 85, wherein the material with higher thermal emissivity comprises PEEK, glazed porcelain, or polypropylene or a combination thereof. Embodiment 87 comprises the method of any one of claims 74 to 86, wherein the thermal fins comprise one or more of: vertical fins, radial fins, cylindrical fins, linear slots, or pin fins. Embodiment 88 comprises the method of claim 87, wherein the pin fins are arranged in a grid pattern. Embodiment 89 comprises the method of any one of claims 74 to 88, wherein the thermal fins comprise at least 1, 2, 3, 4, 5, 6, 7, 8, 9, or 10 thermal fins. Embodiment 90 comprises the method of any one of claims 61 to 89, wherein the imaging device comprises an endoscope.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An imaging device configured for use in a medical procedure or a surgical procedure within a body of an individual;
wherein the imaging device comprises a coating on at least a portion of an exterior of the imaging device; and
wherein the coating comprises a thermal emissivity of about 0.20 to 1.

2. The imaging device of claim 1, wherein the imaging device is configured to dissipate heat at a rate of about 2 to 20 times as compared to a rate of heat dissipation of an imaging device not comprising the coating.

3. The imaging device of claim 1, wherein the imaging device operates at a maximum temperature of about 10° C. to 100° C.

4. The imaging device of claim 1, wherein the imaging device is configured to allow a maximum contact time with the individual undergoing the medical procedure or the surgical procedure that is about 2 to 20 times as compared to a maximum contact time with the individual undergoing the medical procedure or the surgical procedure of an imaging device not comprising the coating.

5. The imaging device of claim 1, wherein the coating comprises a metal or metal alloy.

6. The imaging device of claim 5, wherein the coating comprises the metal.

7. The imaging device of claim 6, wherein the metal is selected from the group consisting of aluminum, magnesium, and titanium.

8. The imaging device of claim 1, wherein the coating comprises polytetrafluoroethylene (PTFE).

9. The imaging device of claim 1, wherein the coating comprises more than one layer.

10. The imaging device of claim 9, wherein the more than one layer comprises an inner layer comprising a conductive material.

11. The imaging device of claim 10, wherein the conductive material comprises aluminum or copper or a combination thereof.

12. The imaging device of claim 9, wherein the more than one layer comprises an outer layer comprising a material with a higher thermal emissivity than an inner layer.

13. The imaging device of claim 12, wherein the material with higher thermal emissivity comprises PEEK, glazed porcelain, or polypropylene or a combination thereof.

14. An imaging device configured for use in a medical procedure or a surgical procedure within a body of an individual;
wherein the imaging device comprises a set of thermal fins disposed on an exterior of the imaging device; and
wherein the set of thermal fins increases a surface area of the exterior of the imaging device by a factor of about 2 to 20 times as compared to an imaging device not comprising the set of thermal fins.

15. The imaging device of claim 14, wherein the imaging device is configured to dissipate heat at a rate of about 2 to 20 times as compared to a rate of heat dissipation of an imaging device not comprising the set of thermal fins.

16. The imaging device of claim 14, wherein the imaging device operates at a maximum temperature of about 10° C. to 100° C.

17. The imaging device of claim 14, wherein the imaging device is configured to allow a maximum contact time with the individual undergoing the medical procedure or the surgical procedure that is about 2 to 20 times as compared to a maximum contact time with the individual undergoing the medical procedure or the surgical procedure of an imaging device not comprising the set of thermal fins.

18. The imaging device of claim 14, wherein the imaging device comprises a coating comprising a metal or metal alloy.

19. The imaging device of claim 18, wherein the coating comprises the metal.

20. The imaging device of claim 19, wherein the metal is selected from the group consisting of aluminum, magnesium, and titanium.

21. The imaging device of claim 14, wherein the imaging device comprises an endoscope.

22. The imaging device of claim 14, wherein the imaging device comprises a polytetrafluoroethylene (PTFE) coating.

23. The imaging device of claim 14, wherein the imaging device comprises a coating comprising more than one layer.

24. The imaging device of claim 23, wherein the more than one layer comprises an inner layer comprising a conductive material.

25. The imaging device of claim 24, wherein the conductive material comprises aluminum or copper or a combination thereof.

26. The imaging device of claim 23, wherein the more than one layer comprises an outer layer comprising a material with a higher thermal emissivity than an inner layer.

27. The imaging device of claim 26, wherein the material with higher thermal emissivity comprises PEEK, glazed porcelain, or polypropylene or a combination thereof.

28. The imaging device of claim 14, wherein the thermal fins comprise one or more of: vertical fins, radial fins, cylindrical fins, linear slots, or pin fins.

29. The imaging device of claim 28, wherein the pin fins are arranged in a grid pattern.

30. The imaging device of claim 14, wherein the thermal fins comprise at least 1, 2, 3, 4, 5, 6, 7, 8, 9, or 10 thermal fins.
